(19) United States
(12) Patent Application Publication
(10) Pub. No.: US 2018/0271863 A1
(43) Pub. Date: Sep. 27, 2018

(54) TARGETING P2 PURINERGIC RECEPTORS TO TREAT HEPATOCELLULAR CARCINOMA (HCC)

(71) Applicant: Baylor College of Medicine, Houston, TX (US)

(72) Inventors: Sundararajah Thevananther, Bellaire, TX (US); Janielle P. Maynard, Baltimore, MD (US)

(73) Assignee: Baylor College of Medicine, Houston, TX (US)

(21) Appl. No.: 15/764,150

(22) PCT Filed: Oct. 24, 2016
(86) PCT No.: PCT/US2016/058447

§ 371 Date: Mar. 28, 2018

Related U.S. Application Data
(60) Provisional application No. 62/245,474, filed on Oct. 23, 2015.

(51) Int. Cl. A61K 31/505; A61K 31/194; A61K 31/675; A61K 45/06; A61P 35/00

(52) U.S. Cl. CPC A61K 31/505; A61K 31/194; A61P 35/00; A61K 45/06; A61K 31/675

(57) ABSTRACT

Embodiments of the present disclosure pertain to methods of inhibiting cancer cells by exposing the cancer cells to a purinergic receptor antagonist that targets one or more purinergic receptors of the cancer cells. The targeted purinergic receptors can include P2 purinergic receptors, such as P2X purinergic receptor subtypes (e.g., P2X3 or P2X5) and P2Y purinergic receptor subtypes (e.g., P2Y2). In some embodiments, the inhibited cancer cells are associated with hepatocellular carcinoma. In additional embodiments, the present disclosure pertains to methods of treating hepatocellular carcinoma in a subject by administering a purinergic receptor antagonist to the subject such that the antagonist targets one or more purinergic receptors of hepatocellular carcinoma cells in the subject. In some embodiments, the subject is a human being suffering from hepatocellular carcinoma.

### CROSS-REFERENCE TO RELATED APPLICATIONS

This application claims priority to U.S. Provisional Patent Application No. 62/245,474, filed on Oct. 23, 2015. The entirety of the aforementioned application is incorporated herein by reference.

### STATEMENT REGARDING FEDERALLY SPONSORED RESEARCH

This invention was made with government support under Grant No. DK069558, awarded by the National Institutes of Health; and Grant No. DK56338, awarded by the National Institutes of Health. The government has certain rights in the invention.

### BACKGROUND

Hepatocellular carcinoma (HCC) is the second leading cause of cancer deaths worldwide. Furthermore, current methods of treating HCC have numerous limitations, including limited efficacy and availability. Therefore, a need exists for more improved and accessible methods of treating HCC. Various aspects of the present disclosure address this need.

### SUMMARY

In some embodiments, the present disclosure pertains to methods of inhibiting cancer cells by exposing the cancer cells to a purinergic receptor antagonist that targets one or more purinergic receptors of the cancer cells. In some embodiments, the purinergic receptor antagonist includes, without limitation, AF-353, A317491, AF-219, Suramin, PPADS, MRS2159, NF449, PSB-1011, NF770, A740003, RB2, MRS2179, MRS2279, MRS2500, MRS2578, NF340, PSB0739, PPTN, and combinations thereof. In some embodiments, the targeted purinergic receptors include P2 purinergic receptors, such as P2X purinergic receptor subtypes (e.g., P2X1, P2X2, P2X3, P2X4, P2X5, P2X6, P2X7, and combinations thereof) and P2Y purinergic receptor subtypes (e.g., P2Y1, P2Y2, P2Y4, P2Y6, P2Y11, P2Y12, P2Y13, P2Y14, and combinations thereof). In some embodiments, the targeted purinergic receptors include, without limitation, P2X5, P2X3, P2Y2, and combinations thereof.

In some embodiments, the cancer cells are associated with hepatocellular carcinoma. In additional embodiments, the present disclosure pertains to methods of treating hepatocellular carcinoma in a subject by administering a purinergic receptor antagonist to the subject such that the antagonist targets one or more purinergic receptors of hepatocellular carcinoma cells in the subject. In some embodiments, the subject is a human being suffering from hepatocellular carcinoma.

The purinergic receptor antagonists of the present disclosure can have various effects on cancer cells. For instance, in some embodiments, the exposure of cancer cells to purinergic receptor antagonists kills the cancer cells. In some embodiments, the exposure of cancer cells to purinergic receptor antagonists reduces or inhibits the proliferation of the cancer cells. In some embodiments, the exposure of cancer cells to purinergic receptor antagonists attenuates ATP-mediated proliferation of the cancer cells. In some embodiments, the attenuated proteins include, without limitation, cyclin D3, cyclin E, cyclin A, Amphiregulin, CTGF, Sox 9, Survivin, and combinations thereof. In some embodiments, the attenuated proteins include Yap-mediated proteins, such as Amphiregulin, CTGF, Sox 9, Survivin, and combinations thereof.

### FIGURES

FIG. 1 provides schemes of methods of inhibiting cancer cells (FIG. 1A) and treating hepatocellular carcinoma (FIG. 1B).

FIG. 2 provides data demonstrating that increased P2X3 purinergic receptor mRNA expression is associated with poor recurrence-free survival in hepatocellular carcinoma (HCC) patients. FIG. 2A shows a qRT-PCR analysis of RNA isolated from HCC tumors, adjacent uninvolved areas and normal livers. Relative mRNA expression was calculated with reference to normal livers. FIG. 2B is a Western Blotting of total proteins isolated from TMC cohort patient tumor (T, 20 μg), uninvolved areas (U, 20 μg), normal liver tissue (N, 20 & 45 μg), and HepG2 cells (20 μg). FIG. 2C is an immunohistochemical analysis of P2X3 expression in normal human liver and TMC cohort liver tumors. FIG. 2D shows a recurrence-free survival analysis (Kaplan-Meier) of Korean patient cohort, including ‘high’ (above median) vs ‘low’ (below median) expression of P2X3 and P2Y13 in HCC tumors (n=188). Distribution of P2X3 and PY13 mRNA expression in TMC patient cohort (n=42) is also shown. FIG. 2E shows the frequency of ‘high’ (≥2-fold) P2 purinergic receptor mRNA expression, as compared to uninvolved areas in HCC tumors of patients with HCV vs. non-viral etiologies. FIG. 2F shows recurrence-free survival analysis (Kaplan-Meier) of Korean patient cohort in HCC tumors (n=156), including ‘low’ P2X3 (below median), HBV positive vs ‘low’ P2X3 (below median), HBV negative vs ‘high P2X3 (above median), HBV positive vs ‘high P2X3 (above median), and HBV negative.

FIG. 3 shows oncomine analysis of P2X3 (Mas Liver dataset) and P2Y13 (Chen Liver dataset) mRNA expression in Hepatocellular Carcinoma vs. normal liver. Data represents log 2 median-centered intensity±SD.

FIG. 4 shows increased P2X3 purinergic receptor protein expression in HCC tumors with viral and non-viral etiologies. Immunohistochemical analysis of P2X3 expression in TMC cohort liver tumors is also shown.

FIG. 5 shows that increased P2X3 purinergic receptor mRNA expression is associated with poor recurrence-free survival, regardless of HBV status. Shown are recurrence-free survival analysis (Kaplan-Meier) of Korean patient cohort, including HBV positive-‘low’ P2X3 (below median) vs ‘high P2X3 (above median) (n=131, FIG. 5A); and HBV negative-‘low’ P2X3 (below median) vs ‘high P2X3 (above median) (n=25, FIG. 5B).

FIG. 6 shows the dysregulation of P2 purinergic receptor expression in HCC cell lines. RNA isolated from Huh7 (FIG. 6A), Hep3B (FIG. 6B), SNU-387 (FIG. 6C), and PLC/PRF/5 (FIG. 6D) were analyzed by qRT-PCR for mRNA expression. Relative expression was calculated with reference to primary hepatocytes isolated from normal healthy adults (n=4). In addition, total RNA isolated from freshly-frozen primary human hepatocytes (healthy adults with no known HCC; n=4) were analyzed by qRT-PCR for all 15 isoforms of P2 purinergic receptors (FIG. 6E). Purinergic receptor mRNA expression was calculated with reference to GAPDH, housekeeping gene.

FIG. 7 shows that extracellular nucleotides induce cell-cycle progression and proliferation in liver cancer cell lines. FIG. 7A shows HCC cells after 24 hours of ATP treatment. Light microscopic images (Huh7, Hep3B, SNU-387-10X; PLC/PRF/5-40×) of BrdU immunostained cells are expressed as a percentage of total cells. Arrows point to nuclei with BrdU staining. FIG. 7B shows HCC cell proliferation after 18 hours of ATP treatment, assessed by MTT assay. FIG. 7C shows human hepatocyte cell proliferation after 18 hours ATP or ATPγS treatment, assessed by MTT assay. FIG. 7D shows total RNA isolated from Huh7 cells treated with ATPγS, ATP or ADP for 24 hours and analyzed by quantitative RT-PCR for cyclin mRNA. FIG. 7E shows temporal expression of cyclin mRNA. FIG. 7F shows Western blotting of total proteins from Huh7 cells after 18, 24 or 30 hours of ATPγS treatment. FIG. 7G shows western blotting of total proteins from human hepatocytes after 24 hours of ATPγS treatment. FIG. 7H shows cyclin mRNA expression in Huh7 cells after 24 hours of nucleotide treatment±pre-treatment (30 minutes) with suramin. Data represented as the mean±SEM, n=3, *p<0.05 to untreated, *p<0.05.

FIG. 8 shows that extracellular nucleotides induce proliferation in Huh7 cells in vitro. Huh 7 cells were maintained in serum-free conditions for 24 hours prior to treatment with ATPγS or ADP for 12, 18 and 24 hours. Light microscopic images (10X) of BrdU immunostained cells are also shown. BrdU-positive cells are expressed as a percentage of total number of cells. Data represents mean±SEM, n=3-6, *p<0.05 vs untreated (un).

FIG. 9 shows that extracellular ATP treatment induces cyclin D3 protein expression in HCC cells in vitro. HCC cells (Huh7, Hep3B, SNU-387, and PLC/PRF/5) maintained in serum-free conditions were treated with ATP for 24 hours and total protein extracts were analyzed by Western blotting for cyclin D3 expression. The α-tubulin was utilized as the protein loading control. Data represents mean±SD, n=3-6, *p<0.05 vs untreated (un).

FIG. 10 shows that extracellular nucleotides induce cell-cycle progression via activation of JNK signaling in Huh7 cells. Shown are Western Blotting of total protein extracts of Huh7 cells after 5, 15 and 30 minutes of ATPγS or ADP treatment (100 μM) (FIG. 10A), and ATPγS (24 hr)±pre-treatment (30 min) of JNK inhibitor, SP600125 (30 μM) (FIG. 10B).

FIG. 11 shows that P2X3 antagonist, AF-353, attenuates ATP-mediated induction of Huh7 cell proliferation. FIG. 11A shows light microscopic images (10X) of BrdU immunostained Huh7 cells after ATP (100 μM, 24 hr)±pre-treatment with AF-353 (5 μM). Arrows point to BrdU-positive cells, expressed as a percentage of total cells. Data represents mean±SEM, n=3-6, *p<0.05 vs. untreated. FIGS. 11B-D shows Western Blotting of total proteins extracted from Huh7 cells and human hepatocytes treated (24 h) with ATP±P2X3 antagonist, AF-353 (5 μM) (FIG. 11B), P2X3 agonist, 2MeSATP (50 μM) (FIG. 11C), ATP±P2X3 antagonist, A317491 (30 μM) (FIG. 11D), and P2X3 antagonist, AF-353 alone (FIG. 11E). Data represents mean±SD, n=3, *p<0.05 vs. untreated, #p<0.05.

FIG. 12 shows that P2X3 antagonist, AF-353, attenuates ATP-mediated activation of Hep 3B cell proliferation, in vitro. Light microscopic images (10X) of BrdU immunostained cells are also shown. Hep3B cells were maintained in serum-free conditions for 24 hours and were pre-treated with AF-353 (5 μM) for 30 minutes, prior to treatment with ATP (100 μM) for 24 hours. BrdU-positive cells are expressed as a percentage of total number of cells. Data represents mean±SEM, n=3-6, *p<0.05 vs untreated (un).

FIG. 13 shows P2X3 overexpression induces basal and ATP mediated proliferation. FIG. 13A shows light microscopic images (10X) of BrdU immunostained Huh7 cells after pCMV6 plasmid or P2X3 DNA transfection±ATP (100 μM, 24 hr)±pre-treatment with AF-353 (5 μM). Arrows point to BrdU-positive cells, expressed as a percentage of total cells. FIG. 13B shows HCC cell proliferation after 72 hours of pCMV6 plasmid or P2X3 DNA transfection, as assessed by MTT assay. Data represents mean±SEM, n=3-6, *p<0.05 vs. untreated.

FIG. 14 shows oncomine analysis of Cyclin D3 (Mas Liver dataset) mRNA expression in Hepatocellular Carcinoma vs normal liver. Data represents log 2 median-centered intensity±SD.

FIG. 15 shows overexpression of P2X3 RNA in Huh7 cells. RNA isolated from Huh7 cells after transfection with P2X3 DNA or pCMV6 vector control plasmids (1 μg) for 24 hours were analyzed by qRT-PCR for P2X3 mRNA. Data represented as the mean±SEM, n=4, *p<0.05 vs. untreated.

FIG. 16 shows that P2 purinergic receptors were dysregulated in Mst1/2^{-/-} mice HCC tumors. Total RNA isolated from Mst1/2^{-/-} mice livers (1 month, 3 month, and 6⁺ months) and age matched WT mice were analyzed by qRT-PCR for P2X (FIG. 16A) and P2Y (FIG. 16B) purinergic receptor mRNA expression. FIG. 16C shows a western blot that analyzes the expression of P2X2, P2X5, P2X7 and P2Y2. The data is represented as the mean±SEM (FIGS. 16A-B) or SD (FIG. 16C), n=3, *p<0.05 vs. WT, ^{#}p=0.05.

FIG. 17 shows P2X3 overexpressed in HCC mouse tumors. Shown are immunohistochemical analyses of P2X3 expression in Mst1/2^{-/-} livers with age-matched WT mice livers (FIGS. 17A-B), and B. CAR; β-Catenin activated mice (8 months) with age matched WT controls (FIG. 17C).

FIG. 18 provides data indicating that P2X5 overexpression correlates to lower HCC patient survival. FIG. 18A shows data relating to recurrence free survival and overall survival analysis (Kaplan-Meier) of Korean patient cohort, including ‘high’ (above median) vs. low (below median) expression of P2X5 in HCC tumors (n=188). FIG. 18B shows the distribution of P2X5 receptor mRNA expression in TMC patient cohort (n=42). FIG. 18C shows oncomine analysis of P2X5 Mas Liver dataset comparing Hepatocellular Carcinoma, normal liver, cirrhosis and liver cancer precursors. The data represents log 2 median-centered intensity±SD.

FIG. 19 shows that P2Y2 underexpression correlates with lower HCC patient survival. FIG. 19A shows data relating to recurrence free survival and overall survival analysis (Kaplan-Meier) of Korean patient cohort, including ‘high’ (above median) vs. low (below median) expression of P2Y2 in HCC tumors (n=188). FIG. 19B shows the distribution of P2Y2 receptor mRNA expression in TMC patient cohort (n=42). FIG. 19C shows oncomine analysis of P2Y2 Mas Liver dataset comparing Hepatocellular Carcinoma, normal liver, cirrhosis and liver cancer precursor. Data represents log 2 median-centered intensity±SD.

FIG. 20 shows that extracellular nucleotides induce Yap nuclear expression and Yap target genes. Shown are western blots of total proteins from Huh7 cells treated with ATPγS, ATP, and UTP for 5, 15 and 30 min for p-MST1/2 (FIG. 20A); with ADP for 5, 15 and 30 min for p-MST1/2 and p-LATS (FIG. 20B); and with ATP, ADP and ATPγS (12 h) for p-YAP and YAP (FIG. 20C). FIG. 20D shows western blots of nuclear extracts from ATPγS and ATP (24 h) treated Huh7 cells for YAP. FIGS. 20E-F show the qRT-PCR analyses of total RNA from ATPγS, ATP and ADP±P2 purinergic receptor antagonist PPADS (24 h) treated Huh7 cells for Amphiregulin, CTGF, Sox9 and survivin mRNA expression. FIG. 20G shows western blotting done on total proteins isolated from Huh7 cells treated with ATPγS or ATP±P2X3 antagonist AF-353 for survivin expression. Data is represented as the mean±SEM (FIGS. 20A-B) or SD (FIG. 20C), n=3, *p<0.05 vs. untreated, ^{#}p<0.05.

FIG. 21 shows data relating to Yap target genes (i.e., Amphiregulin, CTGF, Sox9 and survivin) overexpressed in HCC tumors. FIG. 21A shows the distribution of high (≥2-fold) and low (≤0.5 fold) expression in TMC Cohort patients. FIG. 21B shows the overall survival analysis (Kaplan-Meier) of Korean patient cohort, including ‘high’ (above median) vs. low (below median) expression of survivin and P2Y2/survivin in HCC tumors (n=188).

### DETAILED DESCRIPTION

It is to be understood that both the foregoing general description and the following detailed description are illustrative and explanatory, and are not restrictive of the subject matter, as claimed. In this application, the use of the singular includes the plural, the word “a” or “an” means “at least one”, and the use of “or” means “and/or”, unless specifically stated otherwise. Furthermore, the use of the term “including”, as well as other forms, such as “includes” and “included”, is not limiting. Also, terms such as “element” or “component” encompass both elements or components comprising one unit and elements or components that comprise more than one unit unless specifically stated otherwise.

The section headings used herein are for organizational purposes and are not to be construed as limiting the subject matter described. All documents, or portions of documents, cited in this application, including, but not limited to, patents, patent applications, articles, books, and treatises, are hereby expressly incorporated herein by reference in their entirety for any purpose. In the event that one or more of the incorporated literature and similar materials defines a term in a manner that contradicts the definition of that term in this application, this application controls.

Hepatocellular carcinoma (HCC) is the second leading cause of cancer deaths worldwide. The incidence and deaths resulting from HCC have steadily increased in the US over the past three decades. Moreover, it is predicted that incidence and deaths resulting from HCC will continue to rise.

Currently, the prognosis for HCC is unfavorable. In particular, HCC has an overall survival rate of about 3% to about 7%.

Treatment options for HCC are also limited. In particular, liver resection or orthotopic liver transplantation (OLT) have been the preferred approaches for treating HCC. However, such methods have numerous limitations.

For instance, resection has a very high recurrence rate of up to 70% at 5 years. While OLT has a better prognosis, most patients are not suitable candidates for OLT. Furthermore, a scarcity of organs contributes to long wait periods. In particular, more than 16,000 patients were on the waiting list for livers in 2012.

As such, more improved and accessible methods are required for treating HCC. Various embodiments of the present disclosure address this need.

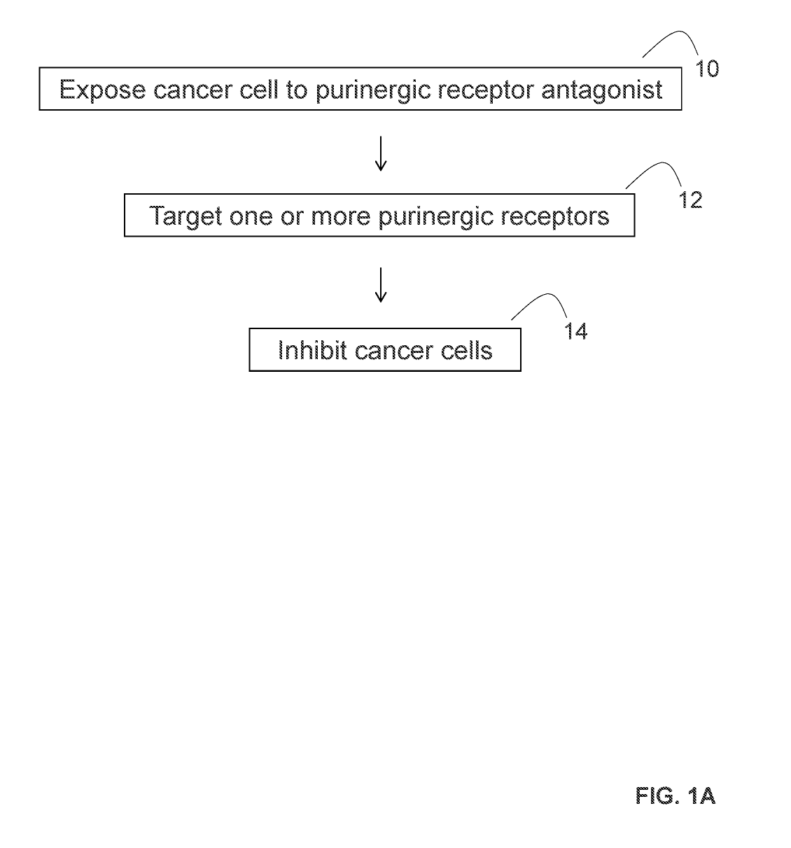

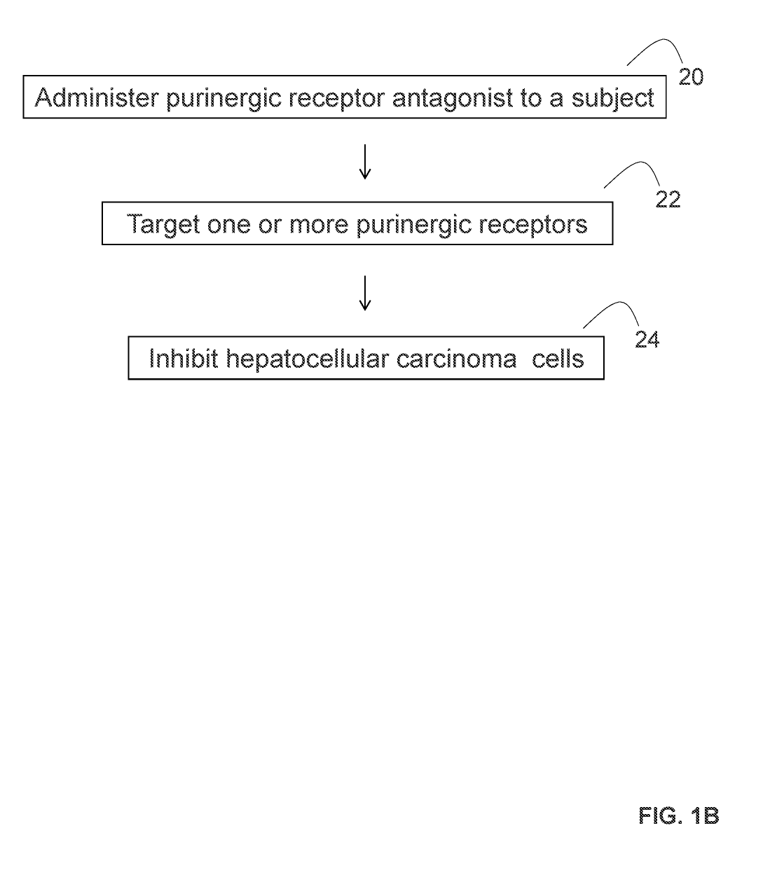

In some embodiments, the present disclosure pertains to methods of inhibiting cancer cells. In some embodiments illustrated in FIG. 1A, such methods involve exposing the cancer cells to a purinergic receptor antagonist (step 10). In some embodiments, the purinergic receptor antagonist targets one or more purinergic receptors of the cancer cells (step 12). In some embodiments, the targeting results in the inhibition of the cancer cells (step 14).

In additional embodiments, the present disclosure pertains to methods of treating hepatocellular carcinoma in a subject. In some embodiments illustrated in FIG. 1B, such methods involve administering a purinergic receptor antagonist to the subject (step 20). In some embodiments, the purinergic receptor antagonist targets one or more purinergic receptors of hepatocellular carcinoma cells in the subject (step 22). In some embodiments, the targeting results in the inhibition of the hepatocellular carcinoma cells in the subject (step 24).

As set forth in more detail herein, the methods of the present disclosure may utilize various purinergic receptor antagonists that target various types of purinergic receptors. Moreover, various methods may be utilized to expose various types of cancer cells to the purinergic receptor antagonists. Furthermore, the purinergic receptor antagonists may inhibit cancer cells and treat hepatocellular carcinoma in various subjects through various mechanisms.

Purinergic Receptor Antagonists

The methods of the present disclosure may utilize various purinergic receptor antagonists. For instance, in some embodiments, the purinergic receptor antagonists include, without limitation, AF-353, A317491, AF-219, Suramin, PPADS, MRS2159, NF449, PSB-1011, NF770, A740003, RB2, MRS2179, MRS2279, MRS2500, MRS2578, NF340, PSB0739, PPTN, and combinations thereof. In some embodiments, the purinergic receptor antagonist is A317491. In some embodiments, the purinergic receptor antagonist is AF-353. In some embodiments, the purinergic receptor antagonist is PPADS. Additional purinergic receptor antagonists can also be envisioned.

The purinergic receptor antagonists of the present disclosure may be in various forms. For instance, in some embodiments, the purinergic receptor antagonists of the present disclosure may be in solid form, liquid form, gaseous form, or combinations of such forms.

In some embodiments, the purinergic receptor antagonists of the present disclosure are part of a therapeutic composition. Such therapeutic compositions may have various components.

For instance, in some embodiments, the therapeutic compositions of the present disclosure include a carrier. In some embodiments, the carrier includes, without limitation, carbon-based nanomaterials, liposomes, polymers, micelles, microspheres, nanostructures, dendrimers, and combinations thereof.

In some embodiments, the therapeutic compositions of the present disclosure also include formulation materials for modifying, maintaining, or preserving various parameters (e.g., pH, osmolarity, viscosity, clarity, color, isotonicity, odor, sterility, stability, rate of dissolution, rate of release, rate of adsorption, rate of penetration, and combinations thereof). In some embodiments, suitable formulation materials can include, without limitation, amino acids (e.g., glycine), antimicrobials, antioxidants (e.g., ascorbic acid), buffers (e.g., Tris-HCl), bulking agents (e.g., mannitol and glycine), chelating agents (e.g., EDTA), complexing agents (e.g., hydroxypropyl-beta-cyclodextrin), and combinations thereof.

The therapeutic compositions of the present disclosure may be in various forms. For instance, in some embodiments, the therapeutic compositions of the present disclosure may be in the form of a liquid, a solid, a gas, and combinations thereof. In some embodiments, the therapeutic compositions of the present disclosure may be in the form of a liquid, such as a syrup. In some embodiments, the therapeutic compositions of the present disclosure may be in the form of a solid, such as a pill.

Purinergic Receptors

The purinergic receptor antagonists of the present disclosure may target various types of purinergic receptors associated with cancer cells. For instance, in some embodiments, the targeted purinergic receptors include P2 purinergic receptors. In some embodiments, the targeted purinergic receptors include P2X purinergic receptor subtypes. In some embodiments, the targeted P2X purinergic receptor subtypes include, without limitation, P2X1, P2X2, P2X3, P2X4, P2X5, P2X6, P2X7, and combinations thereof. In some embodiments, the targeted purinergic receptors include P2X5. In some embodiments, the targeted purinergic receptors include P2X3. In some embodiments, the targeted purinergic receptors include P2X2.

In some embodiments, the targeted purinergic receptors include P2Y purinergic receptor subtypes. In some embodiments, the targeted P2Y purinergic receptor subtypes include, without limitation, P2Y1, P2Y2, P2Y4, P2Y6, P2Y11, P2Y12, P2Y13, P2Y14, and combinations thereof. In some embodiments, the targeted purinergic receptors include P2Y2. In some embodiments, the targeted purinergic receptors include P2Y6.

Cancer Cells

The methods of the present disclosure may inhibit various types of cancer cells. For instance, in some embodiments, the targeted cancer cells are associated with liver tumors. In some embodiments, the targeted cancer cells are associated with hepatocellular carcinoma. In some embodiments, the targeted cancer cells are hepatocellular carcinoma cells. In some embodiments, the targeted cancer cells include, without limitation, Huh7, Hep3B, SNU-387, PLC/PRF/5, HepG2, and combinations thereof.

Exposure of Cancer Cells to Purinergic Receptor Antagonists

Various methods may be utilized to expose cancer cells to purinergic receptor antagonists. For instance, in some embodiments, the exposure occurs by incubating the cancer cells with the purinergic receptor antagonist. In some embodiments, the exposure occurs in vitro. In some embodiments, the exposure occurs in vivo in a subject. In some embodiments, the exposure occurs by administering the purinergic receptor antagonist to the subject. In some embodiments, the subject is suffering from hepatocellular carcinoma, and the method is utilized to treat the hepatocellular carcinoma in the subject.

Administration of Purinergic Receptor Antagonists to Subjects

Various methods may also be utilized to administer purinergic receptor antagonists to subjects. For instance, in some embodiments, the administration occurs by methods that include, without limitation, oral administration, inhalation, subcutaneous administration, intravenous administration, intraperitoneal administration, intramuscular administration, intrathecal injection, topical administration, central administration, peripheral administration, and combinations thereof. In some embodiments, the administration occurs by intravenous administration. In some embodiments, the administration occurs by central administration. In some embodiments, the administration occurs by peripheral administration.

Subjects

The methods of the present disclosure may be utilized to treat various subjects. For instance, in some embodiments, the subject is a human being. In some embodiments, the subject is a non-human animal. In some embodiments, the non-human animal includes, without limitation, mice, rats, rodents, mammals, cats, dogs, monkeys, pigs, cattle and horses. In some embodiments, the subject is suffering from hepatocellular carcinoma.

Mechanism of Action

The purinergic receptor antagonists of the present disclosure can have various effects on cancer cells. For instance, in some embodiments, the purinergic receptor antagonists of the present disclosure kill the cancer cells. In some embodiments, the purinergic receptor antagonists of the present disclosure reduce or inhibit the proliferation of the cancer cells. In some embodiments, the purinergic receptor antagonists of the present disclosure antagonize purinergic receptor function in the cancer cells. In some embodiments, the purinergic receptor antagonists of the present disclosure dysregulate purinergic receptor signaling in the cancer cells. In some embodiments, the purinergic receptor antagonists of the present disclosure attenuate ATP-mediated proliferation of the cancer cells.

In some embodiments, the purinergic receptor antagonists of the present disclosure dysregulate purinergic receptor expression and function in the cancer cells. In some embodiments, the purinergic receptor antagonists of the present disclosure attenuate ATP-mediated protein expression in the cancer cells. In some embodiments, the attenuated proteins include, without limitation, cyclin D3, cyclin E, cyclin A, Amphiregulin, CTGF, Sox 9, Survivin, and combinations thereof.

In some embodiments, the purinergic receptor antagonists of the present disclosure attenuate Yap-mediated protein expression in the cancer cells, such as Amphiregulin, CTGF, Sox 9, Survivin, and combinations thereof. In some embodiments, the purinergic receptor antagonists of the present disclosure attenuate ATP-mediated protein expression of survivin.

### Additional Embodiments

Reference will now be made to more specific embodiments of the present disclosure and experimental results that provide support for such embodiments. However, Applicants note that the disclosure herein is for illustrative purposes only and is not intended to limit the scope of the claimed subject matter in any way.

### Example 1. P2X3 Purinergic Receptor Overexpression is Associated with Poor Recurrence-Free Survival in Hepatocellular Carcinoma Patients

P2 purinergic receptors are overexpressed in certain cancer tissues, but the pathophysiologic relevance of purinergic signaling in hepatocellular carcinoma (HCC) remains unknown. HCC can be caused by numerous factors. For instance, over 75% of HCC are caused by viral infections such as Hepatitis C Virus (HCV). In addition, 70-90% of all tumor development in HCC is associated with chronic liver injury, inflammation and cirrhosis.

In particular, it is known that liver injury with cellular stress and inflammation is a potent trigger for ATP release from hepatocytes and other liver cells. It is also known that extracellular ATP via the activation of cell surface P2 purinergic receptors influences cell proliferation, differentiation and apoptosis. Moreover, Applicants have previously shown that extracellular ATP-mediated P2 purinergic receptor activation promotes cell cycle progression and proliferation in rat primary hepatocytes via c-Jun N-terminal Kinase (JNK) pathway in vitro and hepatocyte proliferation in response to 70% partial hepatectomy in vivo.

Extracellular ATP-mediated activation of P2X (ligand gated ion channels) and P2Y (G protein-coupled) receptors have been reported to influence cell proliferation, migration or apoptosis of various cancer cell types. Studies suggest that extracellular ATP-mediated activation of P2Y2 receptors promotes proliferation and migration in HCC cells. However, the role of the remaining 14 P2 receptor isoforms in HCC is currently unknown. For instance, Table 1 summarizes the frequency (%) of HCC patients with high mRNA expression of P2 purinergic receptor subtypes as compared to the uninvolved liver or normal liver.

TABLE 1
Purinergic receptor expression in HCC patients in the TMC Cohort.

| | High | High |
|---|---|---|
| | Expression (%) | Expression (%) |
| Receptor Subtype | (vs uninvolved) | (vs normal) |
| P2X3 | 60 | 79 |
| P2Y14 | 48 | 67 |
| P2Y2 | 43 | 40 |
| P2Y6 | 43 | 79 |
| P2X6 | 40 | 52 |
| P2Y4 | 40 | 77 |
| P2X1 | 36 | 52 |
| P2X2 | 36 | 83 |
| P2X4 | 36 | 38 |
| P2X5 | 36 | 60 |
| P2X7 | 33 | 67 |
| P2Y1 | 33 | 43 |
| P2Y11 | 33 | 55 |
| P2Y12 | 33 | 43 |
| P2Y13 | 31 | 40 |

Ectonucleotidases such as CD39 decrease extracellular nucleotide concentrations by hydrolyzing nucleotides to nucleosides and ultimately adenosine. Deletion of Cd39 in mice is shown to increase hepatocyte proliferation and promote hepatocarcinogenesis. Furthermore, P2Y2 mRNA and protein expression are increased in human HCC cells compared to normal hepatocytes. Moreover, others have shown that there is increased P2Y2 and P2Y4 receptor expression in non-HCC cancers.

Recently, peritumoral P2X7 purinergic receptor expression has been associated with poor survival in HCC patients after surgical resection. However, P2 purinergic receptor expression and its role in hepatocyte cell cycle progression in human HCC remain unexplored.

In this Example, Applicants examined the role of P2 purinergic signaling in the pathogenesis of HCC and characterized extracellular nucleotide effects on HCC cell proliferation. Two independent HCC patient cohorts were analyzed for P2 purinergic receptor expression. In addition, nucleotide treated HCC cell lines were evaluated for effects on proliferation and cell cycle progression.

Applicants' results in this Example suggest that multiple P2 purinergic receptor isoforms are overexpressed in liver tumors, as compared to uninvolved liver. In addition, the results indicate that dysregulation of P2 purinergic receptor expression is apparent in HCC cell lines, as compared to human primary hepatocytes.

In addition, Applicants have observed in this Example that high P2X3 purinergic receptor expression is associated with poor recurrence-free survival (RFS), while high P2Y13 expression is associated with improved RFS. Applicants also observed that extracellular nucleotide treatment alone is sufficient to induce cell cycle progression via activation of JNK signaling, and extracellular ATP-mediated activation of P2X3 receptors promotes proliferation in HCC cells. In sum, the results in this Example identify a novel role for dysregulation of P2 purinergic signaling in the induction of hyper-proliferative HCC phenotype and identify P2X3 purinergic receptors as new targets for therapy.

### Example 1.1. Increased P2 Purinergic Receptor mRNA Expression in Human HCC Livers

To determine if P2 purinergic receptor expression is dysregulated in HCC livers, Applicants analyzed 42 pairs of HCC livers (uninvolved vs. tumor) and 6 normal donor liver samples from the TMC cohort. Twenty-one patients were infected with Hepatitis C Virus (HCV), 5 patients with Hepatitis B Virus (HBV) and 10 patients with non-viral etiologies. Information on the etiology was unavailable for 6 patients (Table 2).

TABLE 2
Clinical and pathological features of HCC patients (TMC cohort)
(AJCC, American Joint Committee on Cancer; HBV, hepatitis B
virus; HCV, hepatitis C virus; NA, data not available).

| Variable | | TMC Cohort |
|---|---|---|
| Number of Patients | | 42 |
| | male | 24 (57%) |
| | female | 12 (29%) |
| | NA | 6 (14%) |
| Age | Median | 57 y |
| | Range | 14-76 y |
| Viral status | HCV | 21 (50%) |
| | HBV | 5 (12%) |
| | non viral | 10 (24%) |
| | NA | 6 (14%) |
| Cirrhosis | Yes | 25 (60%) |
| | No | 9 (21%) |
| | NA | 8 (19%) |
| Vasculature Invasion | Yes | 8 (19%) |
| | No | 24 (57%) |
| | NA | 10 (24%) |
| Histological Grade | Well differentiated | 3 (7%) |
| | Well to moderately differentiated | 6 (14%) |
| | Moderately differentiated | 18 (43%) |
| | Moderately to poorly differentiated | 3 (7%) |
| | Poorly differentiated | 1 (2%) |
| | Undifferentiated | 0 (0%) |
| | NA | 11 (26%) |
| AJCC Stage | I | 12 (29%) |
| | II | 15 (36%) |
| | III | 5 (12%) |
| | IV | 0 (0%) |
| | NA | 10 (24%) |

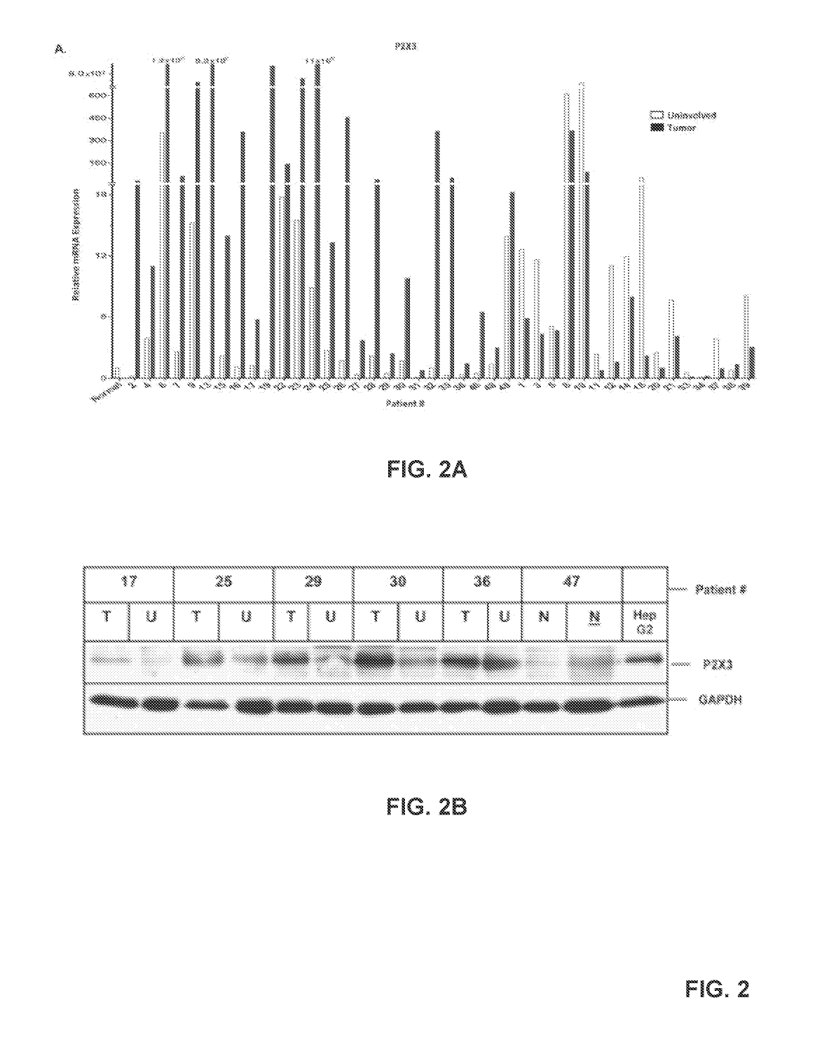

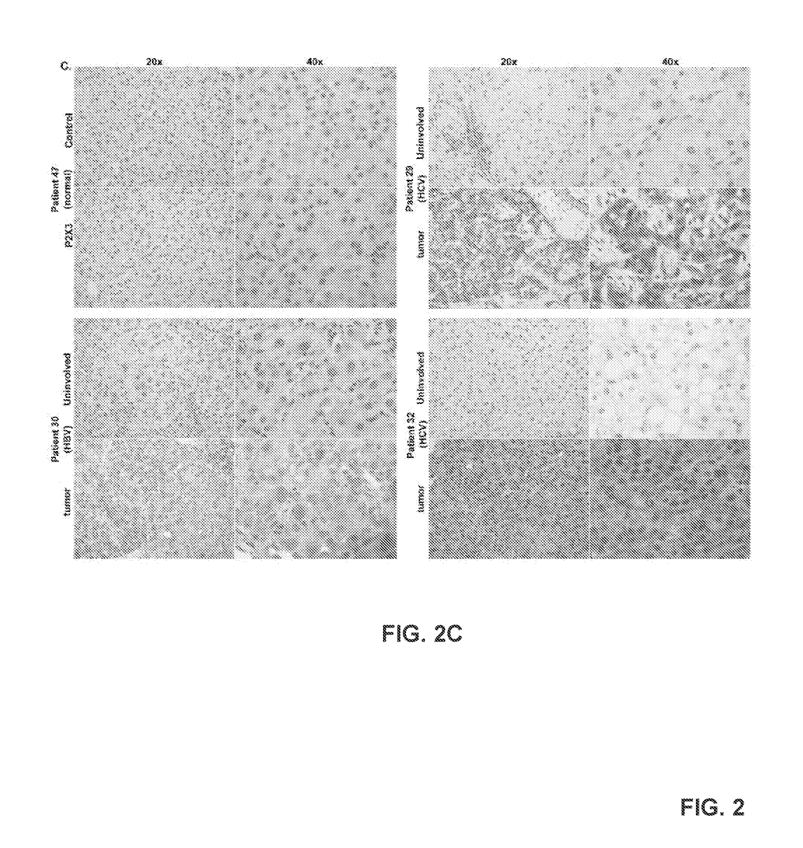

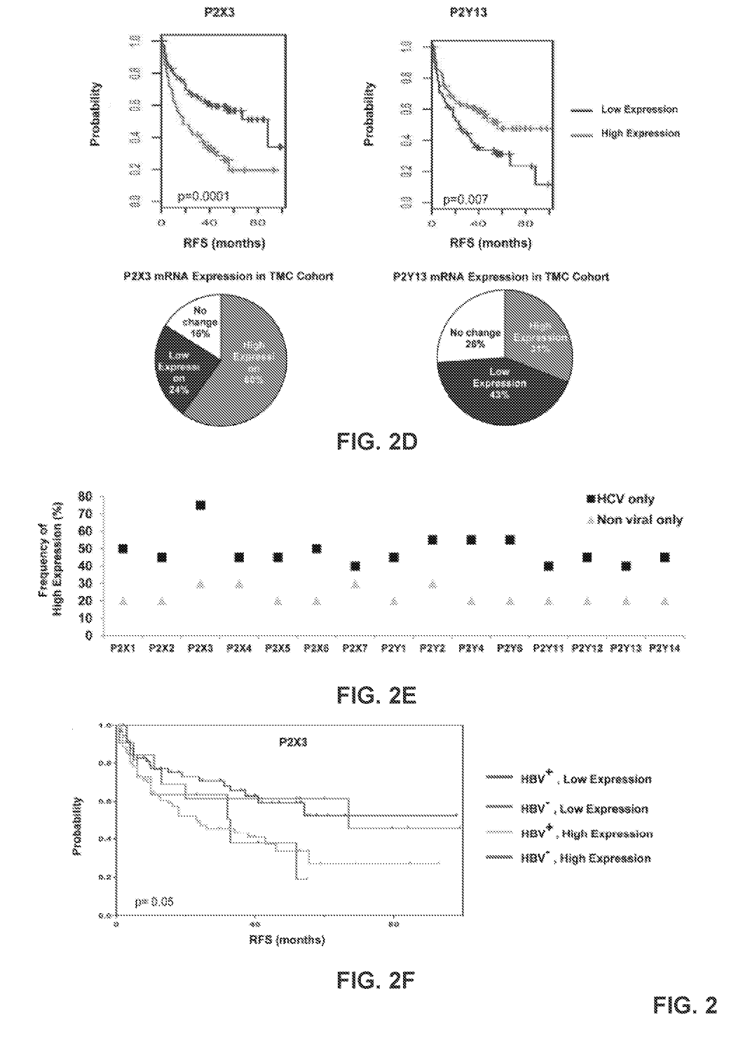

Relative expression of all 15 P2 purinergic receptor isoforms was analyzed by qRT-PCR. Multiple P2 purinergic receptor isoforms were elevated ≥2-fold in liver tumors (‘high’ expression) as compared to uninvolved areas of the liver (Table 1). Applicants' results reveal that 31% of patients in the TMC cohort exhibited ‘high’ expression of at least one P2 purinergic receptor isoform, while 60% of patients exhibited ‘high’ P2X3 mRNA expression in liver tumors, compared to their uninvolved areas (FIG. 2A). P2X3 protein overexpression was observed in HCC tumors compared to uninvolved livers by Western blotting of total homogenates (FIG. 2B). HepG2 cell total protein was used as a positive control.

Further validation was done by immunohistochemical analysis of tumors, uninvolved areas of HCC patient livers, and normal livers. Such analyses revealed that P2X3 protein overexpression was predominant in hepatocytes (FIG. 2C). P2 purinergic receptor mRNA expression was significantly elevated in HCC tumors, as compared to uninvolved areas; despite apparent dysregulation of P2 purinergic receptor expression in the uninvolved areas of most of the patient livers, as compared to normal livers (Table 1). These findings suggest that dysregulation of P2 purinergic receptor expression in chronic liver injury may precede HCC development.

### Example 1.2. P2 Purinergic Receptor Expression Correlates with HCC Patient Survival

The upregulation of multiple P2 purinergic receptor isoforms observed in HCC prompted Applicants to question the significance of dysregulation of P2 purinergic receptor expression on HCC survival in a larger Korean cohort (188 patients). Demographic and pathologic features of this Korean cohort are presented in Table 3.

TABLE 3
Clinical and pathological features of HCC patients (Korean Cohort)
(AJCC, Joint Committee on Cancer; AFP, α-fetoprotein;
BCLC, Barcelona Clinic Liver Cancer; HBV, hepatitis B virus;
NA, data not available)

| | Korean |
|---|---|
| Variable | Cohort |
| Number of patients | 188 |
| Sex, no. (%) | |
| Male | 156 (83%) |
| Female | 32 (17%) |
| NA | |
| Age at baseline, median (range) | 56 y (25-77 y) |
| AFP >300 ng/ml at baseline, no. (%) | |
| Yes | 55 (29%) |
| No | 132 (70%) |
| NA | 1 (1%) |
| HBV at baseline, no. (%) | |
| Yes | 131 (70%) |
| No | 25 (13%) |
| NA | 32 (17%) |
| AJCC stage at baseline, no. (%) | |
| I | 103 (55%) |
| II | 30 (16%) |
| III | 55 (29%) |
| IV | 0 (0%) |
| NA | |
| BCLC stage at baseline, no. (%) | |
| 0 | 4 (2%) |
| A | 106 (56%) |
| B | 63 (34%) |
| C | 11 (6%) |
| D | 4 (2%) |
| NA | |
| Number of deaths | 60 |
| Median follow-up time | 39.6 mo |

Patients were stratified according to ‘high’ (above median) and ‘low’ (below median) P2 purinergic receptor expression based on microarray gene expression analysis of resected livers. Patients with ‘high’ P2X3 expression had a significantly lower recurrence-free survival (RFS) rate than those patients with ‘low’ P2X3 receptor expression (p=0.0001). On the other hand, patients with high P2Y13 expression had significantly improved recurrence free survival (p=0.007) (FIG. 2D). Recall that in the TMC cohort P2X3 was observed as the receptor with the greatest frequency of ‘high’ expression in HCC tumor samples (60%) and P2Y13 was identified as the receptor with the lowest frequency of ‘high’ expression (31%) (FIG. 2D).

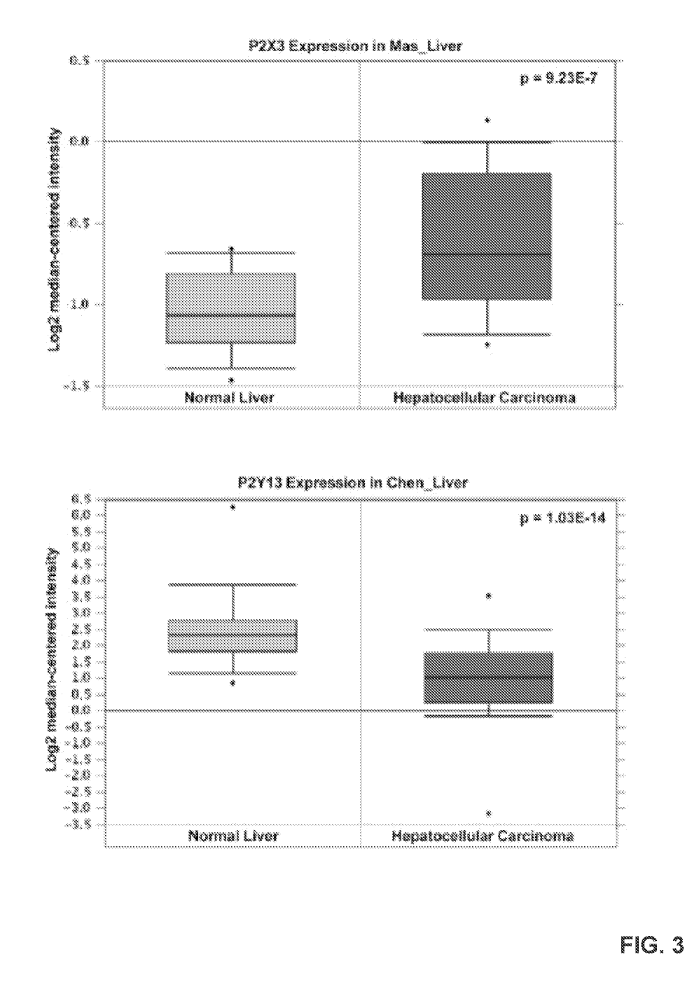

Corroborating Applicants' findings, Oncomine analysis revealed that P2X3 mRNA is significantly overexpressed in the Mas_Liver dataset (p=9.23E-7), while P2Y13 mRNA is underexpressed in the Chen Liver dataset (p=1.03E-14) (FIG. 3). It is noteworthy that P2X3 was ranked among the top 7% overexpressed genes in the Mas_Liver dataset, which included exclusively HCV-positive HCC samples and HCV-negative normal livers. In the TMC cohort, 43% of patients exhibited ‘low’ P2Y13 mRNA expression, which was ranked among the top 4% underexpressed genes in Chen_Liver dataset, and included HCC samples of viral and non-viral origin. Dysregulation of P2X3 and P2Y13 mRNA expression was evident in HCC livers, despite comparable DNA copy numbers of these genes between HCC and normal in the TCGA, Guichard_Liver and Guichard_Liver 2 DNA datasets.

### Example 1.3. P2 Purinergic Receptor Upregulation is More Frequent in HCV Patients

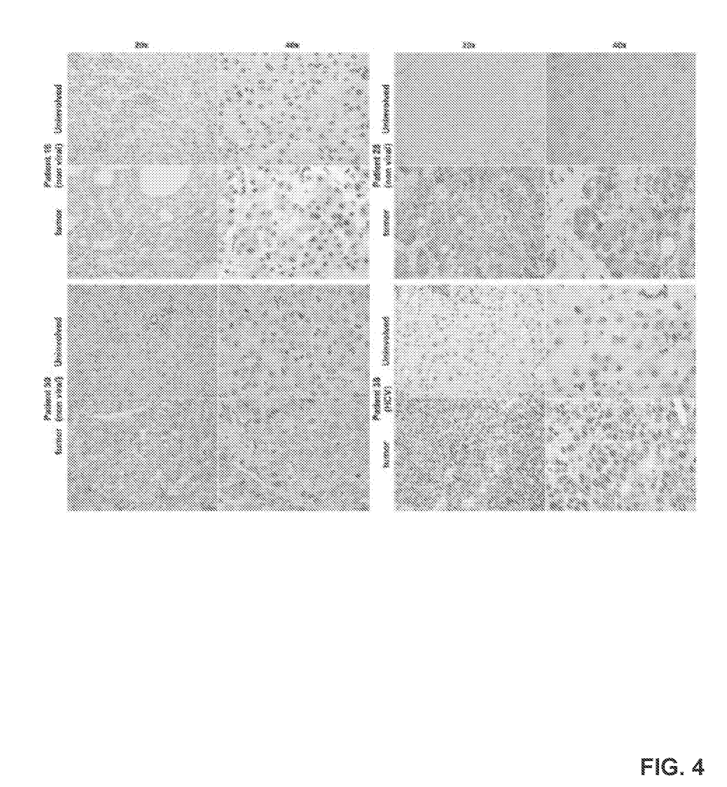

In order to gain insight into the etiology of HCC and its influence on P2 purinergic receptor overexpression in HCC livers, Applicants stratified the TMC patients based on their history of viral infection and found that P2 purinergic receptor upregulation, as assessed by qRT-PCR, was more prevalent among those patients infected with HCV, as compared to non-viral groups (FIG. 2E). P2X3 overexpression is evident in 75% of HCV patients as compared to only 30% of non-viral patients. Immunohistochemical analysis of patient livers with HBV (patient #30), HCV (patient #29, 32, 36) and non-viral patients (patient #16, 28, 50) indicates that P2X3 overexpression in HCC tumors as compared to uninvolved livers can be detected in HCC livers, irrespective of etiology (FIGS. 2C and 4).

To determine whether viral infection influences recurrence free survival, Kaplan-Maier analysis was done on the Korean cohort. Patients were stratified as HBV positive or negative AND P2X3 ‘high’ or ‘low’, as previously defined. Regardless of HBV status, patients with ‘high’ P2X3 expression had lower RFS than patients with ‘low’ P2X3 expression (FIG. 2F).

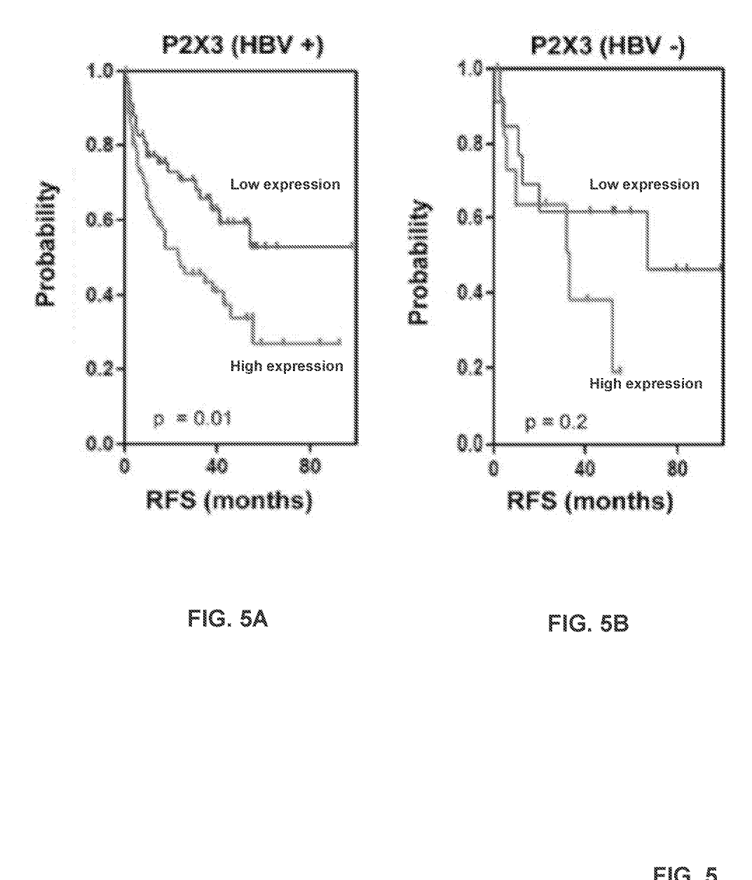

Among HBV positive patients, those with high P2X3 expression had significantly lower RFS compared to those with low P2X3 expression (FIG. 5A). While there was a considerable difference in median survival between P2X3 high (33 months) and P2X3 low (67 months) among HBV viral patients, there was no statistically significant difference in RFS between the two, likely due to small sample size (25 patients) (FIG. 5B).

To determine whether the impact of P2X3 overexpression on risk of recurrence was dependent of HBV status, Cox proportional hazard model analysis was performed. Applicants' data reveals that there was no significant interaction between P2X3 expression and HBV status (p=0.90), confirming that the effect of P2X3 high expression on the risk of recurrence does not significantly differ across HBV groups.

### Example 1.4. Dysregulation of P2 Purinergic Receptor Expression in HCC Cells

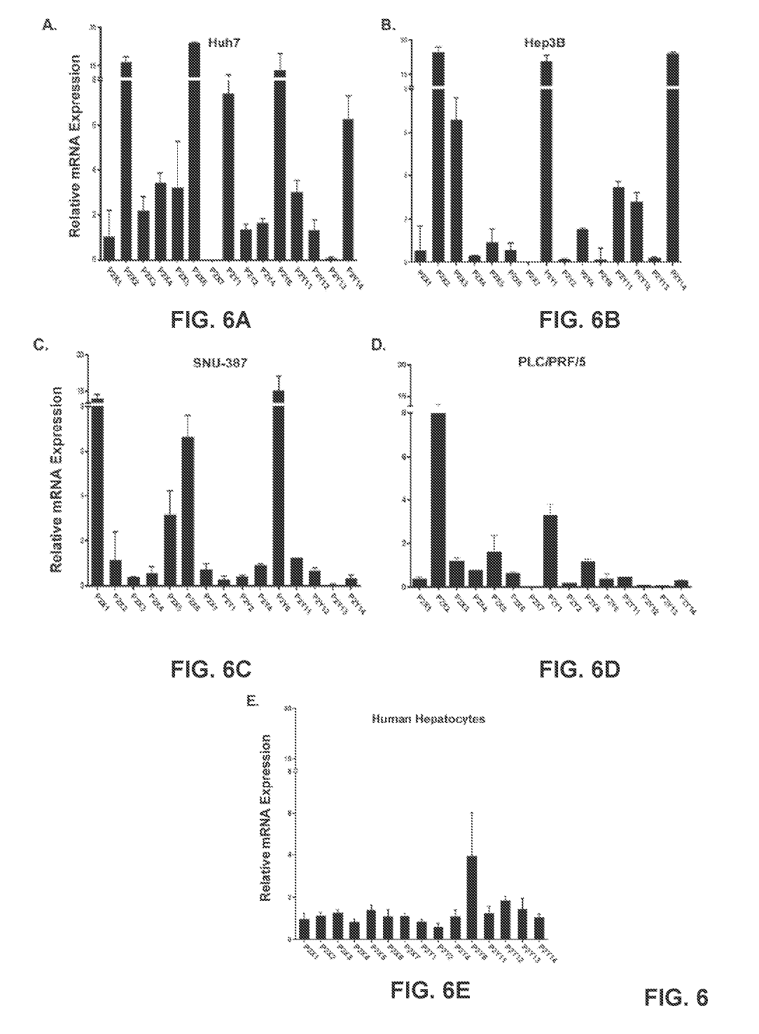

To determine if dysregulation of P2 purinergic receptor expression is associated with hepatocyte transformation to HCC phenotype and to identify a suitable in vitro model system for mechanistic studies, Applicants analyzed P2 purinergic receptor mRNA expression by qRT-PCR in four human hepatocellular carcinoma cell lines-Huh7, Hep3B, SNU-387, PLC/PRF/5, and normal human primary hepatocytes. Suggesting a role for P2 purinergic receptors in hepatocyte transformation, all four HCC cell lines had upregulation of multiple P2 purinergic receptor isoforms and lower expression of P2Y13. Huh7, Hep3B and PLC/PRF/5 had lower expression of P2X7 and upregulation of P2X2 (FIGS. 6A-D). Relative mRNA expression of all 15 purinergic receptor isoforms was comparable in normal human primary hepatocytes (FIG. 6E).

### Example 1.5. Extracellular Nucleotides Induce Proliferation and Cell-Cycle Progression in HCC Cells

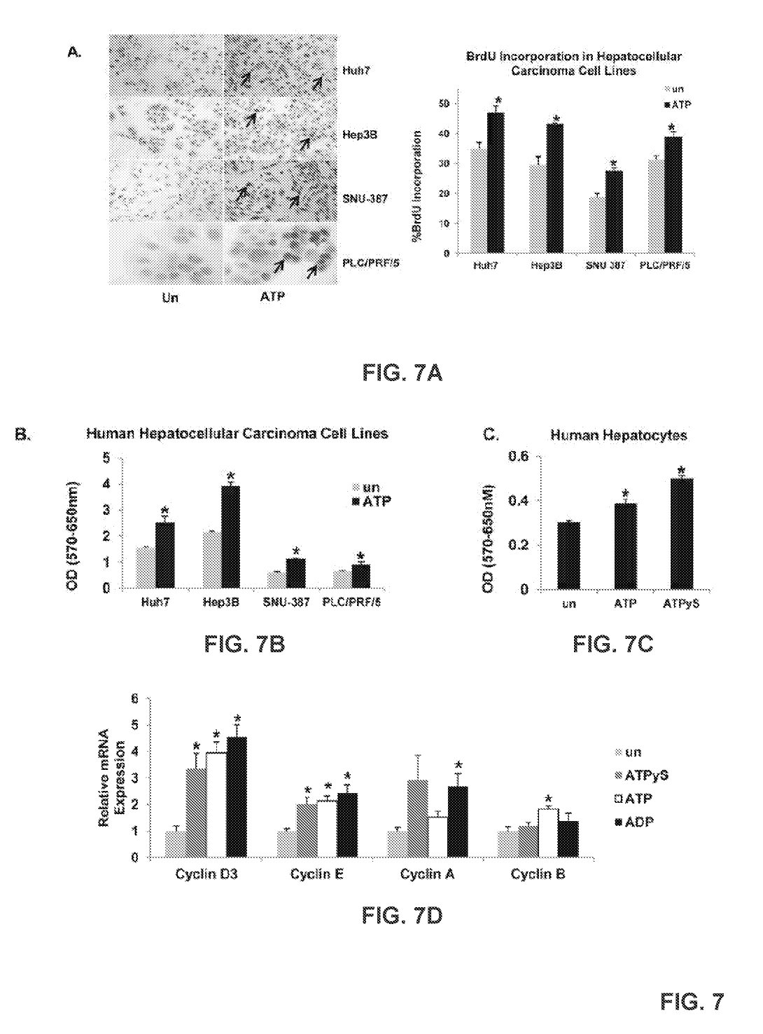

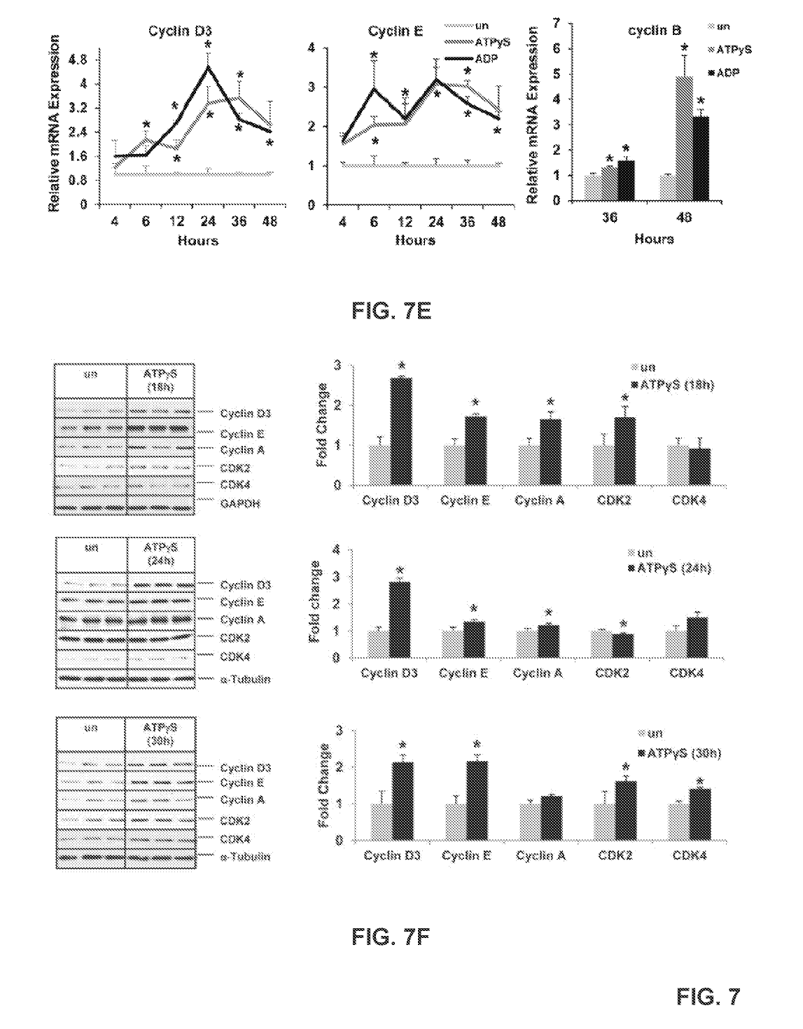

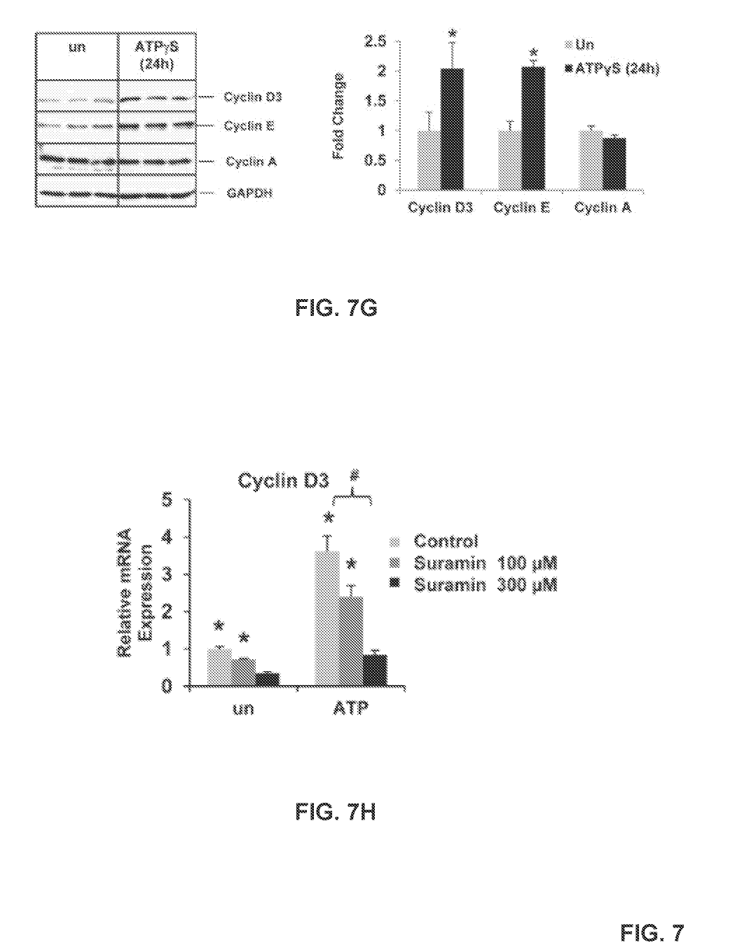

To determine whether P2 purinergic receptor activation influences HCC cell proliferation, BrdU incorporation was assessed in HCC cells maintained in SFM for 24 hours and treated with ATP for 24 hours. ATP treatment alone was sufficient to induce proliferation in each of these cell lines (Huh7, 34%; Hep3B, 46%; SNU-387, 47% and PLC/PRF/5, 24%), identifying nucleotides as liver cancer cell mitogens (FIG. 7A).

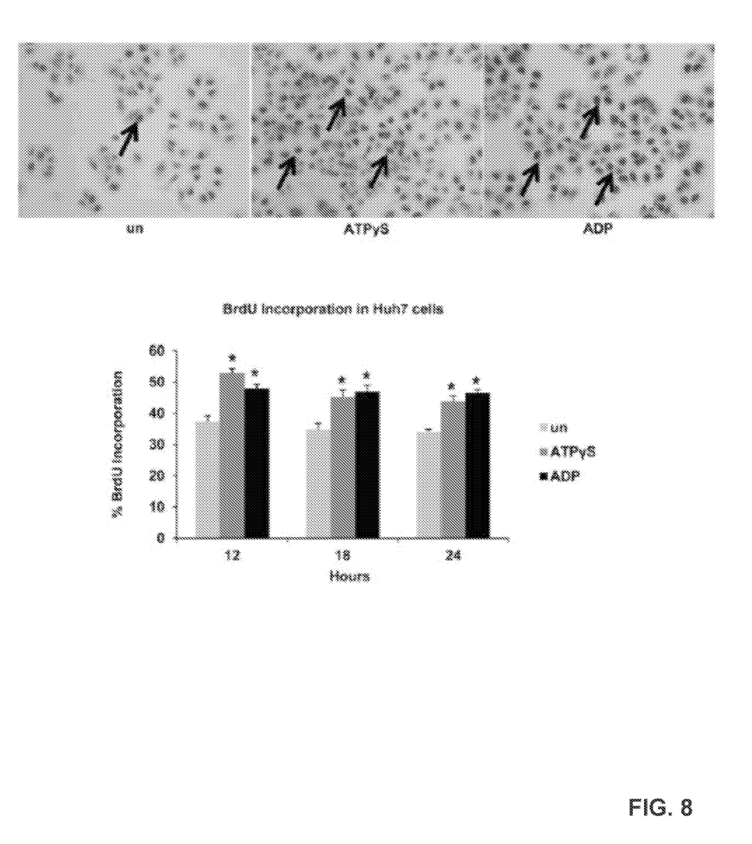

Extracellular ATP can be hydrolyzed to ADP and adenosine by ectonucleotidases at the plasma membrane. Therefore, Applicants tested the effects of ATPγS (non-hydrolyzable analog of ATP) or ADP (break down product of ATP) (100 μM) on Huh7 cell proliferation. ATPγS treatment alone was sufficient to induce BrdU incorporation as early as 12 hours (42%); maintained at 18 h (30%) and 24 h (29%) in Huh7 cells (FIG. 8). ADP was also able to independently increase the number of BrdU positive nuclei (12 h, 30%; 18 h, 35%; 24 h, 37%) (FIG. 8). Furthermore, ATP treatment (18 h) increased cell proliferation and viability in all four cell lines, as determined by MTT assay (Huh7, 65%; Hep3B, 81%; SNU-387, 90% and PLC/PRF/5, 43%) (FIG. 7B). In human hepatocytes, ATP treatment (18 h) led to a modest increase in cell proliferation and viability (27%) while ATPγS treatment (18 h) was more potent (64%) (FIG. 7C).

Nucleotide treatment resulted in increased cyclin D3, cyclin E, cyclin A and cyclin B mRNA expression, correlating with increased proliferation (FIG. 7D). Cyclin D3 mRNA expression was observed after 6 h (ATPγS-2.1 fold), 12 h (ATPγS-1.8 fold; ADP-2.7 fold), 24 h (ATPγS-3.4 fold; ADP-4.5 fold), 36 h (ATPγS-3.5 fold; ADP-2.8 fold) and 48 h (ADP-2.4 fold) treatment, as compared to untreated cells (FIG. 7E). Cyclin E expression exhibited a similar profile while cyclin B expression was induced later at 36 h and 48 h after treatment (FIG. 7E). Western blotting of Huh7 cell total lysates revealed that ATPγS treatment (18 h, 24 h, 30 h) alone was sufficient to induce cyclin D3 (2.7, 2.8, 2.1 fold), cyclin E (1.7, 1.3, 2.2 fold) and cyclin A (1.7, 1.2, 1.2 fold) protein expression (FIG. 7F). ATPγS treatment (30 h) induced CDK2 (1.6 fold) and CDK4 (1.4 fold) protein expression.

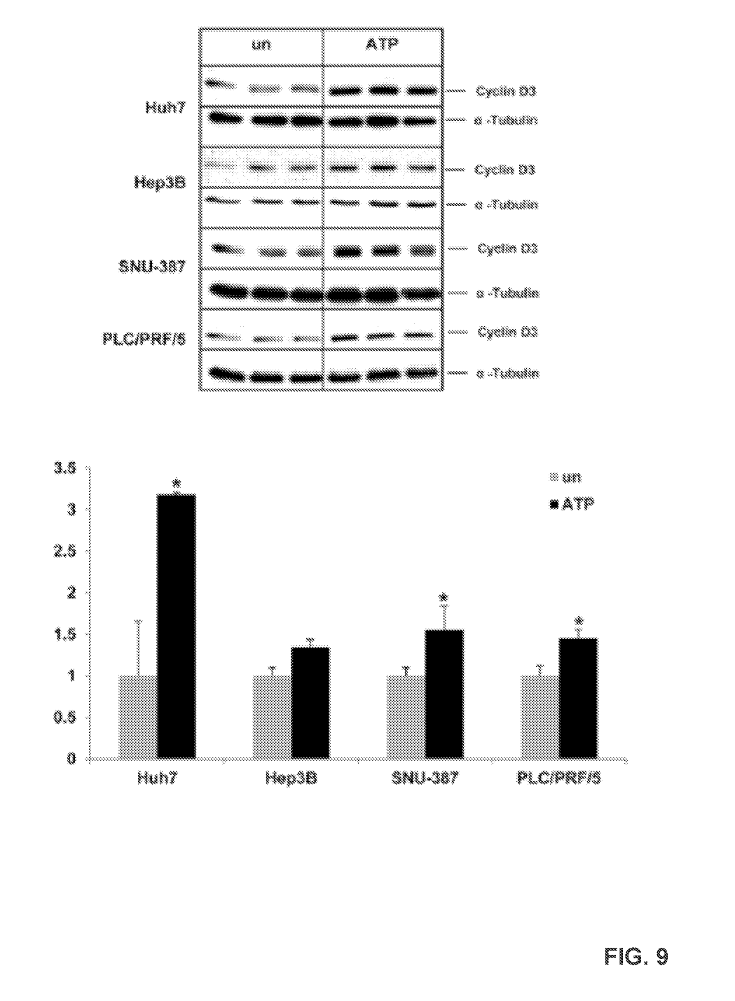

Additionally, ATP treatment alone was sufficient to induce cyclin D3 protein expression in three of the four HCC cell lines tested; Huh7 (3.2 fold), SNU-387 (1.6 fold) and PLC/PRF/5 (1.4 fold)) (FIG. 9). ATPγS treatment (24 h) of human hepatocytes induced cyclin D3 (2.0 fold) and cyclin E (2.0 fold) protein expression (FIG. 7G). These data suggest that nucleotide treatment alone was sufficient to promote cell cycle progression and proliferation in human hepatocytes as well as liver cancer cells.

To confirm that nucleotide effects on dysregulation of cyclin expression is mediated via the activation of P2 purinergic receptors, cells were treated with a broad spectrum P2 purinergic receptor antagonist, Suramin, prior to nucleotide treatment. Suramin pre-treatment resulted in a dose dependent attenuation of cyclin D3 mRNA induction (FIG. 7H). Suramin treatment alone reduced baseline cyclin D3 expression (FIG. 7H), suggesting a role for endogenous ATP release and extracellular nucleotide signaling in basal Huh7 cell cycle progression.

### Example 1.6. Nucleotides Induce Cyclin Expression Via c-Jun N-Terminal Kinase (JNK) Signaling in Huh7 Cells

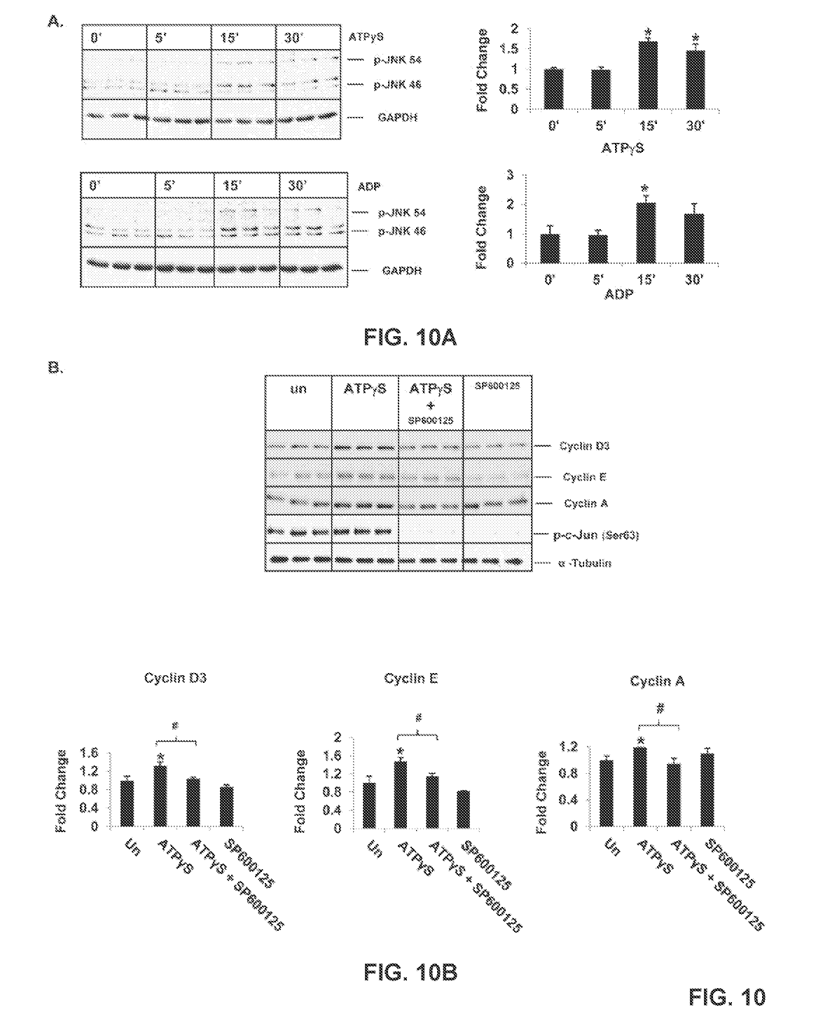

Applicants have previously shown that extracellular nucleotide-mediated activation of JNK signaling induces cell-cycle progression and proliferation of rat primary hepatocytes in vitro. To determine whether extracellular nucleotides induce JNK signaling in transformed hepatocytes, Huh7 cells were treated with nucleotides for 5, 15 and 30 min and total protein extracts were analyzed by Western blotting for JNK phosphorylation (Ser^{Thr/Tyi}). ATPγS treatment alone was sufficient to induce JNK phosphorylation as early as 15 min (1.7 fold) (FIG. 10A). ADP treatment resulted in similar induction (15 min-2 fold).

To determine if extracellular nucleotide-mediated activation of JNK signaling influences cell-cycle progression in transformed hepatocytes, Huh7 cells were treated with SP600125 (JNK inhibitor) prior to ATPγS treatment. Efficiency of JNK inhibition in Huh7 cells was confirmed by Western blotting for p-c-Jun. JNK inhibition completely attenuated ATPγS-mediated induction of cyclin D3, E and A proteins (FIG. 10B), identifying a role for the activation of JNK signaling pathway in P2 purinergic receptor-mediated induced cell-cycle progression in Huh7 cells.

### Example 1.7. P2X3 Antagonism Attenuates Nucleotide-Induced Proliferation and Cell Cycle Progression

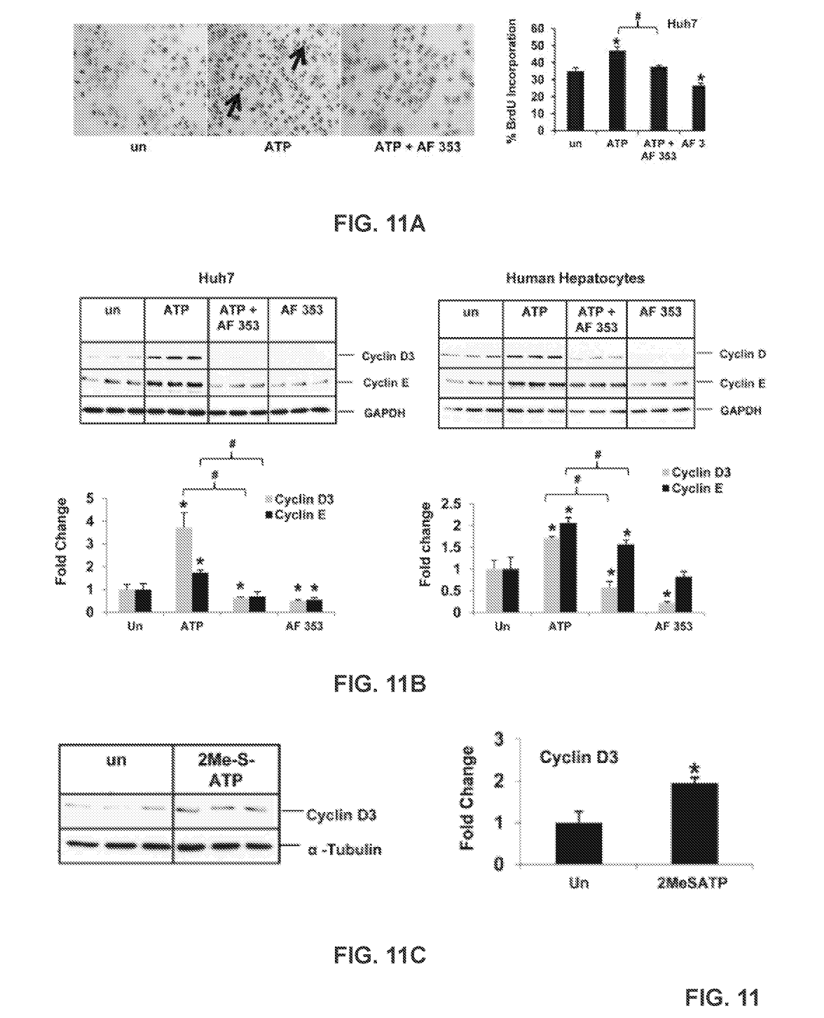

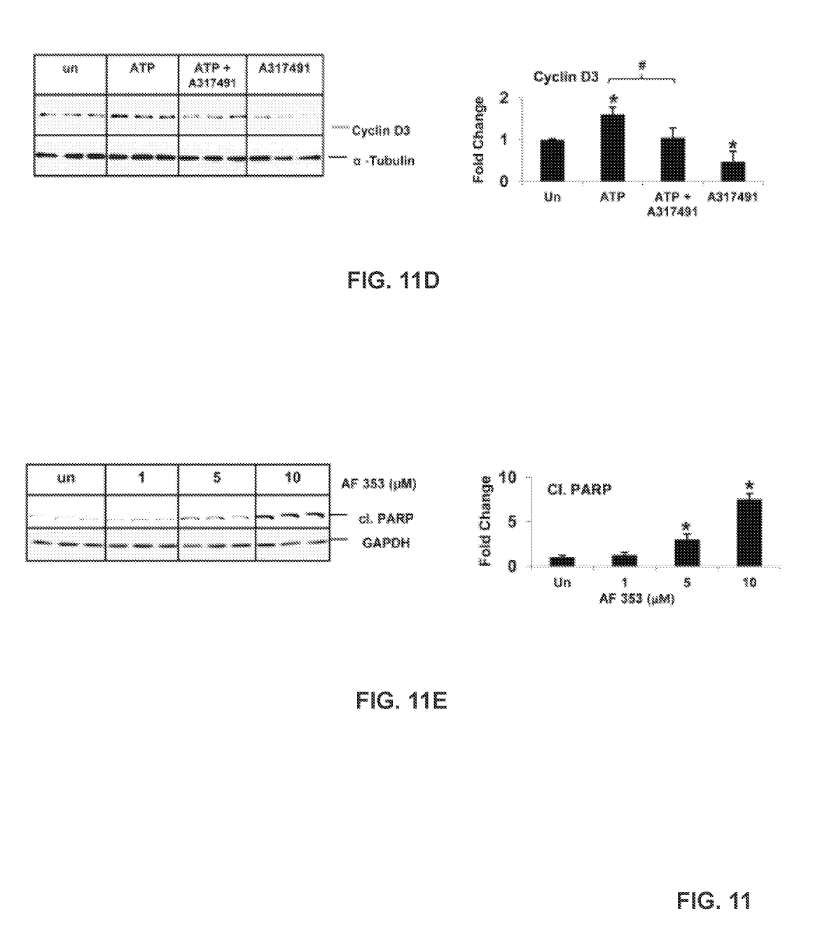

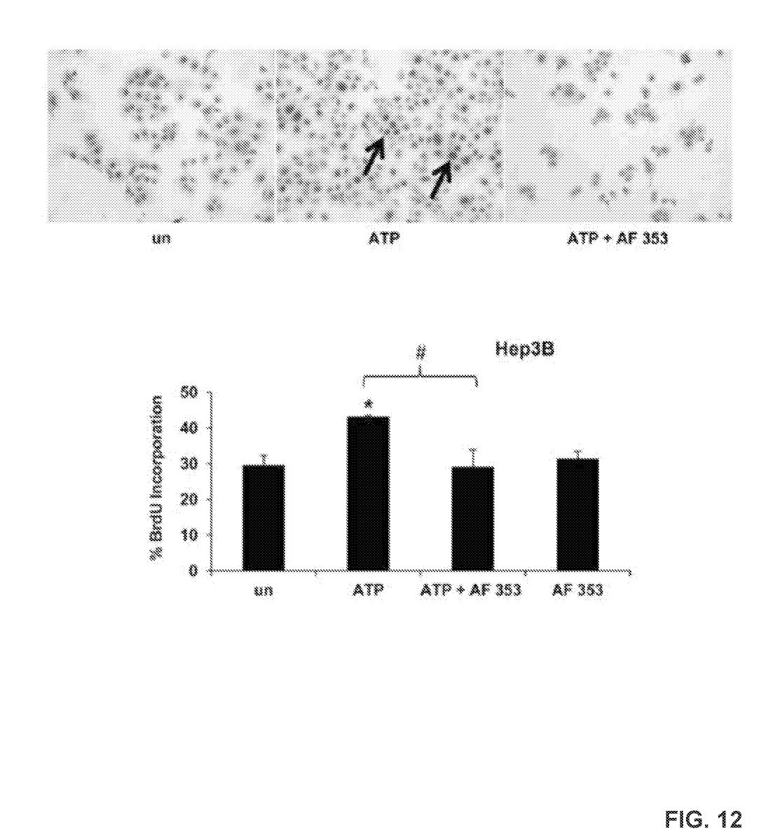

Prompted by the identification of P2X3 as the most frequently overexpressed purinergic receptor isoform in human HCC tumors in Applicants' TMC cohort and that P2X3 overexpression is associated with poor recurrence-free survival in Applicants' Korean cohort, Applicants performed a series of mechanistic studies in Huh7 cells in vitro to determine the functional significance of P2X3 and its role in nucleotide-induced proliferation. Huh7 cells were treated with a highly selective P2X3 antagonist AF-353 prior to ATP treatment. Implicating P2X3 receptors in nucleotide-mediated proliferation, AF-353 pre-treatment completely abolished ATP-mediated increase in BrdU incorporation in Huh7 cells (FIG. 11A). Similarly, ATP-mediated induction of BrdU incorporation in Hep3B cells was completely attenuated by pre-treatment with P2X3 antagonist (FIG. 12). In addition, ATP-mediated induction of cyclin D3 and cyclin E expression were completely attenuated in Huh7 cells and human hepatocytes pre-treated with AF-353 (FIG. 11B).

Applicants' observations that P2X3 agonist 2-MeSATP treatment alone was sufficient to induce cyclin D3 expression (1.9 fold) and pre-treatment with an alternative P2X3 antagonist (A317491) attenuated ATP induced cyclin D3 expression in Huh7 cells further validated the role of P2X3 purinergic receptor activation in the induction of cell-cycle progression in HCC cells (FIGS. 11C-D). Furthermore, P2X3 antagonist treatment alone was sufficient to reduce the baseline BrdU-positive cells and cyclin protein expression in serum-free conditions, implicating P2X3 activation as a necessary facilitator of Huh7 cell cycle progression (FIGS. 11A-B). Additionally, P2X3 antagonist treatment alone resulted in increased cleaved PARP protein expression, an established marker of cells undergoing apoptosis (FIG. 11E).

### Example 1.8. P2X3 Overexpression Increases Basal and ATP Mediated Cell Proliferation

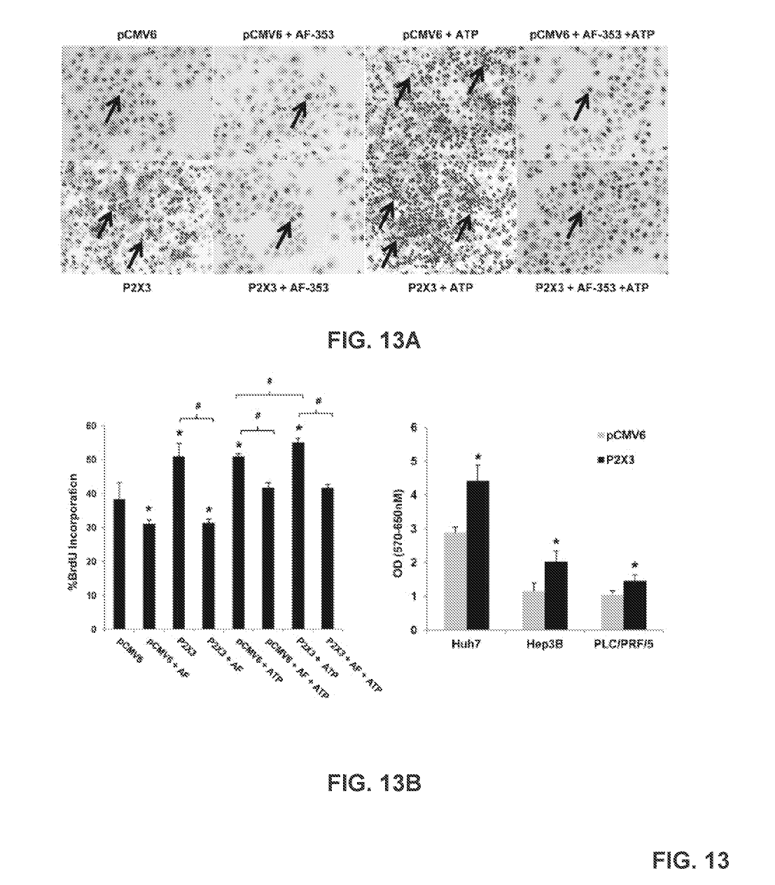

To confirm P2X3 receptor involvement in HCC cell proliferation, P2X3 was overexpressed in Huh7 cells. Thereafter, these cells were treated with ATP+/−P2X3 antagonist, AF-353. BrdU analysis revealed that P2X3 overexpression (72 h post-transfection) alone was sufficient to significantly increase proliferation (51%) when compared to the vector control pCMV6 (38%). Moreover, AF-353 treatment (24 h) completely attenuated the P2X3 mediated increase in proliferation (FIG. 13A).

ATP Treatment (24 h) further increased BrdU incorporation when compared to the pCMV6 control. ATP mediated proliferation was significantly higher in cells overexpressed with P2X3 compared to those transfected with only pCMV6. Again, pre-treatment with AF-353 significantly attenuated the ATP mediated increase in BrdU incorporation. Furthermore, P2X3 overexpression alone was sufficient to improve cell proliferation and viability of Huh7, Hep3B and PLC/PRF/5 cells as assessed by MTT assay (FIG. 13B).

Collectively, the aforementioned findings suggest that P2X3 purinergic receptor expression and function is significant for HCC cell survival and basal proliferation as well as proliferation in response to changes in nucleotide concentrations in extracellular environments.

### Example 1.9. Summary

In sum, Applicants undertook a comprehensive analysis of all 15 P2 purinergic receptor isoforms in HCC tumors, as compared to the adjacent uninvolved areas of HCC livers as well as normal livers. Applicants' studies have identified a distinct dysregulation of P2 purinergic receptor expression in HCC livers. The upregulation of P2 purinergic receptor expression observed among the tumor samples when compared to their adjacent uninvolved areas highlights its significance in the pathogenesis of HCC. Altered P2 purinergic receptor expression in the uninvolved areas of HCC livers (as compared to normal healthy livers) suggests that purinergic signaling may be dysregulated prior to HCC development. Therefore, as potential biomarkers, P2 purinergic receptor upregulation has the advantage of being detected prior to tumor development when effective interventions may be undertaken.

Another significant finding of this study is that there is a higher frequency of P2 purinergic receptor upregulation in HCC tumors of HCV patients, as compared to those tumors with non-viral etiologies, identifying a specific subgroup of HCC with higher prevalence of P2 purinergic receptor overexpression. However, immunohistochemical analysis suggests that P2X3 overexpression is evident in HCC tumors, irrespective of viral status.

Without being bound by theory, it is envisioned that dysregulation of purinergic receptor expression may influence initiation and temporal progression of HCC via its influence on dysregulation of cell-cycle control, a hallmark of HCC cells. In this study, Applicants have identified that P2X3 purinergic receptor expression and function is important for HCC cell survival and proliferation. Moreover, P2X3 was the most frequently upregulated purinergic receptor isoform in Applicants' local TMC patient cohort. It was ranked in the top 7% of overexpressed genes in the Mas_Liver dataset (Oncomine) and its ‘high’ expression had the strongest correlation with decreased recurrence free survival in Applicants' Korean patient cohort. Therefore, P2 purinergic receptors can serve as potential new targets for the development of HCC therapeutics.

While purinergic signaling has been implicated in cancer, its influence on proliferation varies by cancer cell type and cell-type specific P2 purinergic receptor expression profile. Applicants observed differential P2 purinergic receptor expression profiles among the four HCC cell lines tested (FIG. 6). This heterogeneity of purinergic receptor expression profile may dictate varying functional outcomes in cells, in response to nucleotide changes in the extracellular environment. Activation of each receptor isoform elicits unique responses, such that a cancer cell may benefit from increased expression of some isoforms and decreased expression of others.

Confirming previous findings, P2X7 and P2Y13 expression were reduced, as compared to the other receptor isoforms, in three of the four HCC cell lines tested. In Applicants' TMC patient cohort, P2X7 had a lower frequency of upregulation (33%), as did P2Y13 (31%), as compared to other receptors such as P2X3 (60%) (Table 1). Applicants' data show a strong positive correlation between ‘low’ P2Y13 expression and increased recurrence free survival in Applicants' Korean cohort (FIG. 2D).

P2Y13 purinergic receptor, preferentially activated by ADP, has been shown to play a role in the activation of anti-oxidant Nrf2/HO-1 axis to protect against oxidative stress-induced neuronal death. It is well established that oxidative stress contributes to the development of HCC and it is reported that patients with HCC that exhibit increased oxidative stress levels are prone to recurrence after ‘curative’ treatment. P2X2 expression was elevated in three HCC cell lines tested. Compared to the normal livers, P2X2 had the highest frequency of upregulation in HCC tumor tissues (83%) (Table 1). It is noteworthy that P2X2 often forms heteromultimeric complexes with P2X3, the other highly overexpressed receptor in Applicants' TMC patient cohort. Overall, P2 purinergic receptor expression is dysregulated in HCC cells commensurate with its hyper-proliferative transformed phenotype, as compared to quiescent hepatocytes isolated from normal healthy adult livers.

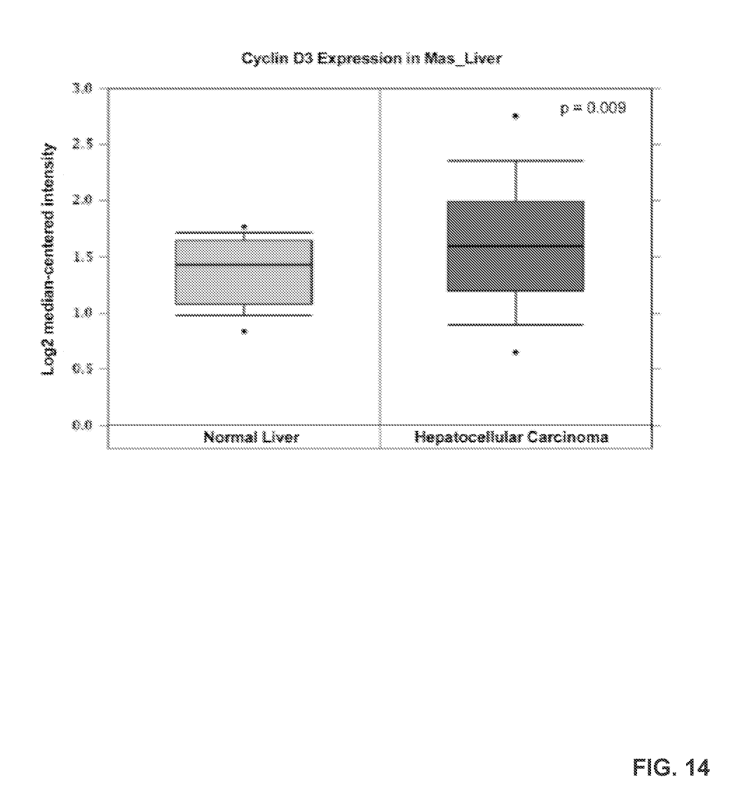

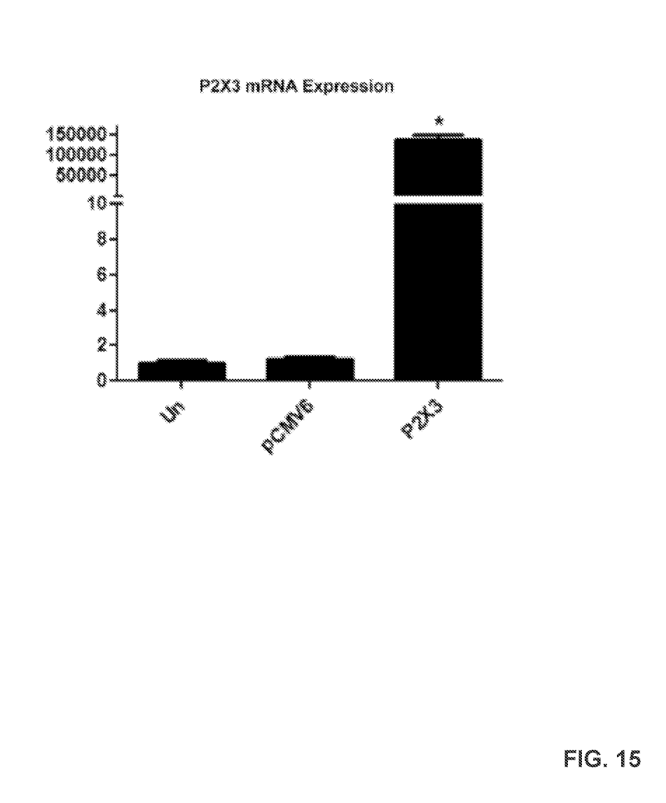

Applicants' findings that extracellular nucleotides induce cyclin D3, cyclin E and cyclin A protein expression via activation of JNK signaling is of significant interest to Applicants' understanding of pathogenesis of HCC, as it has been previously reported that JNK1 expression is increased in primary hepatocellular carcinomas. Cyclin D3 was reported to be upregulated in 51-72% of HCC tissues and was overexpressed in the Mas_liver dataset (FIG. 14). Applicants' in vitro studies suggest nucleotide induced cyclin D3 expression is mediated via activation of P2X3 purinergic receptors in Huh? cells. Applicants show that nucleotide treatment induces CDK4 protein expression. Studies suggest that the function of CDK4 is most critical for G₁/_{S} HCC cell cycle progression and that CDK4 is activated by cyclins D and E.

P2X3 overexpression increases cell proliferation and viability, indicating for the first time the critical role of P2X3 receptors in HCC cell growth. These findings highlight the functional significance of increased P2X3 receptor expression in the tumors. Furthermore, the attenuation of P2X3 mediated proliferation by P2X3 antagonist, AF-353, clearly highlights the potential therapeutic application by pharmaceutical control of P2X3 receptor activation against HCC propagation.

P2X3 receptor expression in afferent neurons and its role in pain sensation is well characterized. Although P2X3 expression has been reported in hepatocytes, cholangiocytes, and portal vein myocyctes in the liver and liver derived cell lines, P2X3 receptor function in hepatic cells has not been well characterized. Recently, P2X3 has been reported to be involved in the complex regulation of liver regeneration through its expression on Natural Killer (NK) cells. It is reported that NK cells are reduced in the intratumoral tissue of HCC patients, particularly those with advanced stage HCC. In this Example, Applicants provide evidence for P2X3 overexpression in hepatocytes, as assessed by immunohistochemical analysis of HCC patient livers and its role in hepatocyte proliferation, as assessed by in vitro studies in human primary hepatocytes and four independent HCC-derived cell lines.

Considering previous findings of increased ATP concentrations in the extracellular environment in injured and inflamed livers and in the tumor interstitium, as well as Applicants' findings of increased P2 purinergic receptor expression in HCC liver tumors, Applicants' observations of nucleotide-mediated increased proliferation in HCC cells is particularly significant. In conclusion, extracellular nucleotides via the activation of P2 purinergic receptors induce proliferation and cell cycle progression in HCC cells. P2 purinergic receptor expression is significantly dysregulated in HCC tumor tissues and exhibits strong correlation with recurrence-free survival in HCC patients. These studies underscore the potential role of purinergic signaling in the pathogenesis of HCC, and highlight the utility of P2 purinergic receptors as a potential new class of biomarkers and therapeutic targets.

### Example 1.10. HCC Patients

Liver tumors and adjacent, uninvolved areas (42 pairs) were obtained from HCC patients undergoing resection or liver transplantation at St. Luke's Episcopal Hospital and Ben Taub Hospital in the Texas Medical Center, Houston Tex. (TMC cohort). Normal livers (6 samples) were obtained from donor livers prior to transplantation at the St. Luke's Episcopal Hospital.

Tumor specimens were obtained from an additional 188 HCC patients undergoing hepatectomy at Seoul National University, Guro Hospital of Korea University, Seoul, Chonbuk National University, Jeonju, and Dong-San Medical Center of Keimyung University, Daegu, Korea (Korean cohort). Gene expression data from the Korean cohort were generated using the Illumina microarray platform human-6 versions 2 and 4 (Illumina, San Diego, Calif.). These patients were followed up prospectively at least once every 3 months after surgery. Primary microarray data from the Korean cohort are available in NCBI's GEO public database (accession numbers GSE16757 and GSE43619). The study protocols were approved by the Institutional Review Boards of institutions, and all participants had provided written, informed consent.

### Example 1.11. Oncomine Database

The oncomine 4.5 (www.oncomine.org), a publicly available database of published cancer gene expression profiles, was queried for alterations in P2 purinergic receptor (P2X3, P2Y13) and cyclin D3 genes with additional filters defined for the analysis type (cancer vs normal) and cancer type: liver cancer. Five gene expression and three DNA copy number datasets were retrieved for further analysis, comparing HCC vs normal. All gene expression data were log-transformed and median-centered and all statistical analyses were performed using functions implemented in Oncomine. P value of less than 0.05 (p<0.05) is considered significant.

### Example 1.12. Hepatocytes, HCC Cell Lines and Gene Transfection

Normal human primary hepatocytes isolated from healthy adults (no known history of HCC) and freshly-frozen prior to shipment (Cryoport Systems, CA) were purchased from Triangle Research Labs, NC. Human hepatocellular carcinoma derived Huh7, Hep3B, SNU-387 and PLC/PRF/5, cell lines were maintained, as described herein.

### Example 1.13. Immunohistochemistry, MTT Assay and Western Blotting

Formalin-fixed and paraffin embedded liver sections from HCC patients were analyzed by immunohistochemistry. HCC cell proliferation was evaluated by immunohistochemical analysis of BrdU incorporation and MTT assay. Total protein extracts of HCC cells were analyzed by Western blotting, as described herein.

### Example 1.14. Real-Time Quantitative Reverse-Transcriptase Polymerase Chain Reaction (qRT-PCR)

Total RNA was isolated from human livers or cells using Trizol Reagent according to manufacturer's instructions (Invitrogen, NY). Complementary DNA (cDNA) synthesis and qRT-PCR were performed, as described herein.

### Example 1.15. Statistical Analysis

Data was analyzed by one way analysis of variance (ANOVA) or unpaired Student's t test. Values of p<0.05 were considered statistically significant. TMC cohort patients were stratified by P2 purinergic receptor expression (‘high’-≥2-fold vs ‘low’-≤0.5 fold as compared to uninvolved areas). Korean cohort patients were stratified according to ‘high’ (above median) and ‘low’ (below median) expression for P2 Receptor gene expression and prognostic difference was assessed by Kaplan-Meier plots and log-rank test. Cox proportional hazard model analysis was done on the Korean patient cohort to test the interaction between HBV status and P2 purinergic receptor expression on the risk of recurrence.

### Example 1.16. Hepatocytes and HCC Cell Lines

Normal human primary hepatocytes isolated from healthy adults (no known history of HCC) in suspension or freshly-frozen prior to shipment (Cryoport Systems, CA) were purchased from Triangle Research Labs, NC. Fresh hepatocytes were plated on collagen-coated tissue culture plates or glass coverslips in Williams E complete media with additives (10% fetal bovine serum, 2 mM glutamine, 2.5 g/ml insulin, 4 ng/ml glucagon, 2.5 g/ml transferrin, 2.5 ng/ml sodium selenite, 10,000 U/ml penicillin, 10,000 g/ml streptomycin, 50 g/ml gentamycin) for 3 h to ensure hepatocyte adherence to plates. Subsequently, hepatocytes were maintained in Williams E minimal media free from serum and growth factors for 24 h prior to treatment. Human hepatocellular carcinoma derived Huh7, and Hep3B, were cultured in Minimum Essential Medium Eagle (MEM); SNU-387 was cultured in Roswell Park Memorial Institute (RPMI) medium and PLC/PRF/5 cultured in Dulbecco's Modified Eagle's medium (DMEM). All maintenance culture media were supplemented with 10% fetal bovine serum (FBS), L-Glutamine (2 mM), penicillin (100 units/ml) and streptomycin (100 mg/ml) at 37° C. and 5% CO₂. Cells were maintained in serum free media (containing 2 mM L-Glutamine, 100 units/ml penicillin and (100 mg/ml)) streptomycin for 24 h prior to treatment.

### Example 1.17. Cell Transfection

Huh7 cells were maintained in MEM with 10% fetal bovine serum (FBS), 5% L-Glutamine and 5% Penicillin-streptomycin overnight. P2X3 or pCMV6 vector control plasmids (1 m) were transfected with Turbofectin 8.00 (Origene Technologies, Rockville, Md.) in MEM with 5% L-Glutamine. Media was replaced after 24 hours, according to manufacturer's instructions.

### Example 1.18. Immunohistochemistry

Formalin-fixed and paraffin embedded liver sections from HCC patients were analyzed by immunohistochemistry with anti-P2X3 antibody (Abcam, Cambridge, Mass.). HCC cells were grown on glass coverslips; BrdU (10 μM, Roche, Indianapolis, Ind.) was added to culture media for 1 h prior to fixation (cold acetone:methanol, 1:1) and stained using anti-BrdU antibody (DAKO, Carpinteria, Calif.) and DAB Peroxidase Substrate Kit (Vector Labs, Burlingame, Calif.), according to manufacturer's instructions. Counterstaining was done with hematoxylin. BrdU positive cells were counted and expressed as a percentage of the total number of cells in ten randomly selected high-power fields (20×) per coverslips.

### Example 1.19. MTT Assay

Cell viability was determined using the 3-(4, 5-dimethylthiazol-2-yl)-2,5-diphenyltetrazolium (MTT, Sigma) assays. Cells were plated in 96-well plates, maintained in serum-free media for 24 h and treated with ATP (100 μM) for 18 h. Cells were exposed to MTT solution (0.7 mg/ml) and incubated at 37° C. for 2 h. The media was removed and 200 μl of dimethyl sulphoxide (DMSO) was added to each well. After shaking the plates for 30 min, the absorbance at 570 nM was measured (background subtraction at 650 nM).

### Example 1.20. Western Blotting

Total protein extracts were obtained by homogenizing cells in total lysis buffer (50 mM Tris-HCl, pH 7.5, 0.5 M NaCl, 2 mM EGTA, 2 mM EDTA, 1.0% Triton X-100, 0.25% Deoxycholate, 1 m/ml pepstatin, 1.0 m/ml leupeptin, 1.0 m/ml aprotinin, 1.0 mM phenylmethylsulfonyl fluoride, 1.0 mM Dithiothreitol, 2.0 mM activated Na₃VO₄, and 2.0 mM NaF) and centrifuging at 14,000 rpm for 10 min (4° C.). Equal amounts of total proteins as determined by BCA protein Assay (Pierce, Rockford, Ill.) were analyzed by Western blotting. Blots were probed with antibody specific for a-tubulin or GAPDH to ensure equal loading of proteins in each lane.

### Example 1.21. Real-Time Quantitative Reverse-Transcriptase Polymerase Chain Reaction (qRT-PCR)

Total RNA was isolated from human livers or cells using Trizol Reagent according to manufacturer's instructions (Invitrogen, NY). Complementary DNA (cDNA) synthesis was performed by reverse transcription of total RNA (2 μg) with high capacity cDNA reverse transcription kit (Applied Biosystems, Foster city, CA). The cDNA product was amplified by qRT-PCR in Step One Plus Real-Time PCR system using SYBR Green PCR Master Mix (Applied Biosystems, Grand Island, N.Y.). Quantitative expression values were determined using the ΔΔACₜ method as specified by manufacturers using GAPDH as a control. DNA sequences of gene-specific primers are listed in Table 4.

TABLE 4
Oligonucleotide sequences.

| Gene | Forward Sequence | Reverse Sequence |
|---|---|---|
| GAPDH | 5′-CGGAGTCAACGGATTTGGTCGTAT-3′ | 3′-AGCCTTCTCCATGGTCGTGAAGAC-3′ |
| P2X1 | 5′-CGCCTTCCTCTTCGAGTATGA-3′ | 5′-AGATAACGCCCACCTTCTTATTACG-3′ |
| P2X2 | 5′-GCCTACGGGATCCGCATT-3′ | 5′-TGGTGGGAATCAGGCTGAAC-3′ |
| P2X3 | 5′-GCTGGACCATCGGGATCA-3′ | 5′-GAAAACCCACCCTACAAAGTAGGA-3′ |
| P2X4 | 5′-CCTCTGCTTGCCCAGGTACTC-3′ | 5′-CCAGGAGATACGTTGTGCTCAA-3′ |
| P2X5 | 5′-CTGCCTGTCGCTGTTCGA-3′ | 5′-GCAGGCCCACCTTCTTGTT-3′ |
| P2X6 | 5′-AGGCCAGTGTGTGGTGTTCA-3′ | 5′-TCTCCACTGGGCACCAACTC-3′ |
| P2X7 | 5′-TCTTCGTGATGACAAACTTTCTCAA-3′ | 5′-GTCCTGCGGGTGGGATACT-3′ |
| P2Y1 | 5′-CGTGCTGGTGTGGCTCATT-3′ | 5′-GGACCCCGGTACCTGAGTAGA-3′ |
| P2Y2 | 5′-GAACTGACATGCAGAGGATAGAAGAT-3′ | 5′-GCCGGCGTGGACTCTGT-3′ |
| P2Y4 | 5′-CCGTCCTGTGCCATGACA-3′ | 5′-TGACCGCCGAGCTGAAGT-3′ |
| P2Y5 | 5′-GCCGGCGACCACATGA-3′ | 5′-GACCCTGCCTCTGCCATTT-3′ |
| P2Y11 | 5′-CTGGAGCGCTTCCTCTTCAC-3′ | 5′-GGTAGCGGTTGAGGCTGATG-3′ |
| P2Y12 | 5′-AGGTCCTCTTCCCACTGCTCTA-3′ | 5′-CATCGCCAGGCCATTTGT-3′ |
| P2Y13 | 5′-GAGACACTCGGATAGTACAGCTGGTA-3′ | 5′-GCAGGATGCCGGTCAAGA-3′ |
| P2Y14 | 5′-TTCCTTTCAAGATCCTTGGTGACT-3′ | 5′-GCAGAGACCCTGCACACAAA-3′ |
| Cyclin D3 | 5′-AGGGATCACTGGCACTGAAG-3′ | 5′-ACAGGTGTATGGCTGTGACAT-3′ |
| Cyclin E | 5′-TGTGTCCTGGATGTTGACTGCC-3′ | 5′-CTCTATGTCGCACCACTGATACC-3′ |
| Cyclin A | 5′-GCACACTCAAGTCAGACCTGCA-3′ | 5′-ATCACATCTGTGCCAAGACTGGA-3′ |
| Cyclin B | 5′-GACCTGTGTCAGGCTTTCTCTG-3′ | 5′-GCTATTTTGGTCTGACTGCTTGC-3′ |

### Example 2. P2 Purinergic Receptor Activation Induces YAP Target Genes Implicated in the Pathogenesis of Hepatocellular Carcinoma

The Hippo kinase pathway has been identified as a potent tumor suppressor in the mammalian liver. The hippo signaling pathway was initially identified in Drosophila as an essential regulator of cell proliferation. Thereafter, the pathway has been shown to regulate mammalian organ size and suppress tumor formation by the repression of cell proliferation and promotion of apoptosis. The hippo pathway upstream kinases Mst1/2 and Lats1/2 are involved in a cascade to phosphorylate the transcriptional activator protein Yap, leading to its cytoplasmic retention. Alternatively, attenuation of Yap phosphorylation results in nuclear translocation, where it is involved in transcription of genes that promote cell proliferation and/or suppress cell death. Specific deletion of Mst1/2 in mouse hepatocytes results in excessive proliferation, causing enlarged livers and by 5-6 months, hepatocellular carcinoma.

As such, Yap has recently been well studied as an oncogene, and has been reported as overexpressed in multiple cancers, including HCC. In fact, 50-85% of HCC tumors are reported to have Yap overexpression, amplification or nuclear translocation. Furthermore, Yap is highly associated with shorter overall survival and disease-free survival; and is determined as an independent prognostic marker in HCC.

Yap function is mediated primarily through its interaction with the TEA domain (TEAD) family of transcription factors. TEAD2, TEAD3 and TEAD4 are most potently activated by Yap and share a strong physical interaction with Yap compared to other transcription factors reported to interact with Yap. Yap-transcription factor interactions induce multiple genes implicated in cancer. Yap target genes cyclin E, Amphiregulin (AREG), connective tissue growth factor (CTGF), SRY [sex determining region Y] box 9 (Sox9) and Survivin are all reportedly overexpressed in HCC patient liver samples compared to uninvolved or normal livers.

AREG is a member of the epidermal growth factor (EGF) family known to stimulate proliferation of most cell types via the binding and activation of EGF receptor (EGFR). Moreover, P2Y purinergic receptors may transactivate the well-known oncogenic prognostic factor, EGFR, to induce proliferation via MAP kinase activation. AREG, a secreted protein, has very low levels in the normal liver, but markedly elevated levels are reported during acute liver injury and regeneration. AREG overexpression was reported in 62-86% of HCC patient tumors.

CTGF is a member of the CCN family of genes, a group of cysteine-rich proteins associated with cell surface and extracellular matrix components involved in a myriad of biological functions including migration, cell survival, growth and differentiation. CTGF is most potently induced by transforming growth factor β₁ (TGFβ₁) and is overexpressed in fibrosis. In liver fibrosis, activated hepatic stellate cells (HSCs) are the main source of CTGF production. CTGF overexpression is reported in multiple cancers, including HCC. Moreover, CTGF promotes cell invasion and growth and is an independent prognostic factor for overall survival.

Sox9 is a member of the sox family proteins, a group of transcriptional regulators involved in cell fate determination during development and the establishment and maintenance of stem and progenitor cell pools. It is reported that sox9 is expressed in the liver during development in the progenitor population but its role in the adult liver progenitor cells is still debated. Sox9 overexpression was associated with tumor progression and poor prognosis in HCC.

Survivin is a member of the Inhibitor of apoptosis (IAP) gene family and more recently found to have roles in cell proliferation and regulation of response to cellular stress. It was believed that survivin was predominantly expressed during fetal development, but recent studies suggest survivin expression during liver regeneration in a cell-cycle dependent manner. Survivin overexpression in HCC is frequently reported and is correlated with poor prognosis.

Applicants recently reported that P2 purinergic receptors are differentially expressed in HCC patient tumors compared to their adjacent non-tumor tissue (Oncotarget 2015; 6:41162-41179). Applicants therefore aimed to investigate the role of P2 purinergic signaling in the pathogenesis of HCC in Mst1/2^{-/-}, and to investigate whether purinergic signaling has a direct influence on hippo signaling in vitro.

Applicants' analysis revealed significant dysregulation of P2 purinergic receptor mRNA and protein expression in Mst1/2^{-/-} livers compared to age matched Wild Type (WT) livers. Activation of P2 purinergic receptors by nucleotide treatment alone was sufficient to induce nuclear YAP protein expression in vitro. Furthermore, nucleotide treatment of HCC cells resulted in the upregulation of YAP target genes; cyclin E, AREG, CTGF, Sox9 and survivin. For the first time, Applicants have shown a significant interaction between purinergic signaling and the hippo kinase pathway which may uncover a pathogenic pathway for HCC.

### Example 2.1. HCC Patients

Liver tumors and adjacent, uninvolved areas (42 pairs) were obtained from HCC patients and normal livers (6 samples) were obtained as described recently (Oncotarget 2015; 6:41162-41179). Tumor specimens were obtained from an additional 188 HCC patients undergoing hepatectomy in Korea (Korean cohort) and gene expression data were generated as described recently (Oncotarget 2015; 6:41162-41179).

### Example 2.2. Oncomine Database

The Oncomine 4.5 (www.oncomine.org), a publicly available database of published cancer gene expression profiles, was queried for alterations in P2X5, P2X7 and P2Y2 receptor genes with additional filters defined for the analysis type (cancer vs. normal) and cancer type (liver cancer). All gene expression data were log-transformed and median-centered and all statistical analyses were performed using functions implemented in Oncomine. P value of less than 0.05 (p<0.05) is considered significant.

### Example 2.3. Hepatocytes and HCC Cell Lines

Normal human primary hepatocytes isolated from healthy adults (no known history of HCC) in suspension were purchased from Triangle Research Labs, NC and maintained as described recently (Oncotarget 2015; 6:41162-41179). Human hepatocellular carcinoma derived Huh7 cells were cultured as previously described (Oncotarget 2015; 6:41162-41179).

### Example 2.4. Immunohistochemistry

Formalin-fixed and paraffin embedded liver sections from WT, Mst1/2^{-/-}, CAR; β-Catenin mice were analyzed by immunohistochemistry with anti-P2X3 antibody (Abcam, Cambridge, Mass.).

### Example 2.5. Western Blotting

Total protein extracts were obtained by homogenizing cells in total lysis buffer and analyzed by Western blotting as described recently (Oncotarget 2015; 6:41162-41179).

### Example 2.6. Real-Time Quantitative Reverse-Transcriptase Polymerase Chain Reaction (qRT-PCR)

Total RNA was isolated from human livers or cells and was amplified by qRT-PCR as described recently (Oncotarget 2015; 6:41162-41179).

### Example 2.7. Statistical Analysis

Data was analyzed by unpaired Student's t test. Values of p<0.05 were considered statistically significant. TMC cohort patients were stratified by P2 purinergic receptor expression (‘high’-≥2-fold vs. ‘low’-≤0.5 fold as compared to uninvolved areas). Korean cohort patients were stratified according to ‘high’ (above median) and ‘low’ (below median) expression for P2 Receptor gene expression and prognostic difference was assessed by Kaplan-Meier plots and log-rank test.

### Example 2.8. P2 Purinergic Receptor Expression is Dysregulated in Mst1/2^{-/-} Livers

Applicants have recently reported that P2 purinergic receptor expression is dysregulated in HCC tumor tissue compared to uninvolved or normal liver tissue (Oncotarget 2015; 6:41162-41179). In order to investigate the expression of P2 purinergic receptors in a progressively developing tumor environment, Applicants analyzed the Mst1/2^{-/-} livers of an HCC mouse model at multiple time points.

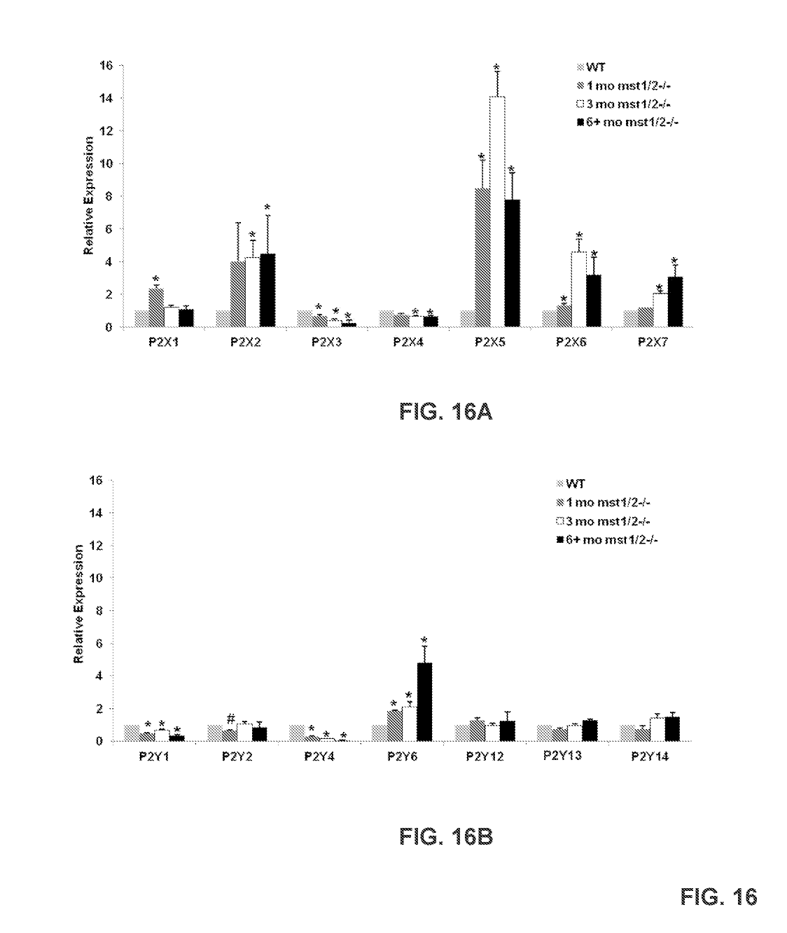

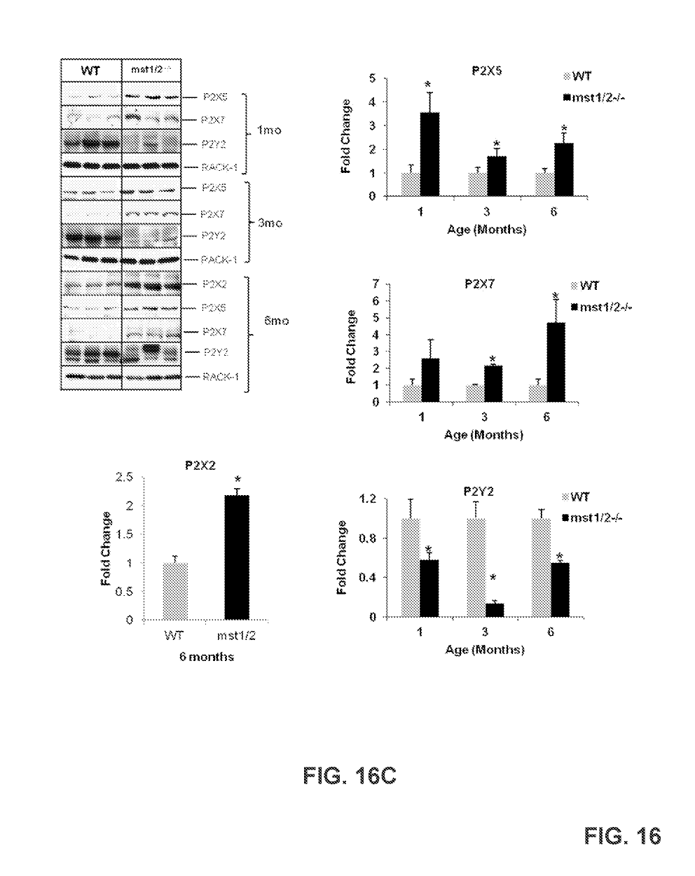

Applicants analyzed the livers of Mst1/2^{-/-} mice at 1 month (mo) (no tumors), 3 mo (1 or 2 nodules) and 6-11 mo (heavily tumor burdened). Comparisons were made to age matched wild type (WT) mice. The temporal profiles show differential expression of both P2X (FIG. 16A) and P2Y (FIG. 16B) purinergic receptor expression in the Mst1/2^{-/-} compared to WT.

P2X5 expression was most dramatically increased among all of the receptor subtypes, with as much as 14-fold mRNA expression in the 3 mo old Mst1/2^{-/-} tumor liver compared to 3 mo old wild type livers (FIG. 16A). Western blotting of mouse liver membrane fractions showed that there was significantly increased P2X5 protein expression in Mst1/2^{-/-} livers compared to WT at 1 mo (3.6 fold), 3 mo (1.7 fold) and 6 mo (2.3 fold) (FIG. 16C).

P2X1, P2X2, P2X6, P2X7 and P2Y6 also had significantly increased mRNA expression while P2X3, P2X4, P2Y1 and P2Y4 showed significant downregulation (FIG. 16). P2X7 mRNA expression appeared to be progressively increased. Statistically significant elevation is observed at 3 months when dysplasia is evident in the Mst1/2^{-/-} and even more at 6 months when the liver is heavily tumor burdened. Western blotting confirmed that P2X7 receptor protein expression was also significantly elevated at 3 mo (2.1 fold) and at 6 mo (4.7 fold) (FIG. 16C).

P2Y2 receptor mRNA expression was mostly comparable among the three time points, with a trend toward downregulation at 1 mo (p=0.05) (FIG. 16B). However, western blotting revealed significantly reduced P2Y2 protein expression at all three ages; 1 mo (0.58 fold), 3 mo (0.13 fold) and 6 mo (0.55 fold) (FIG. 16C) suggesting post transcriptional regulation of P2Y2 expression in the Mst1/2^{-/-} tumor model.

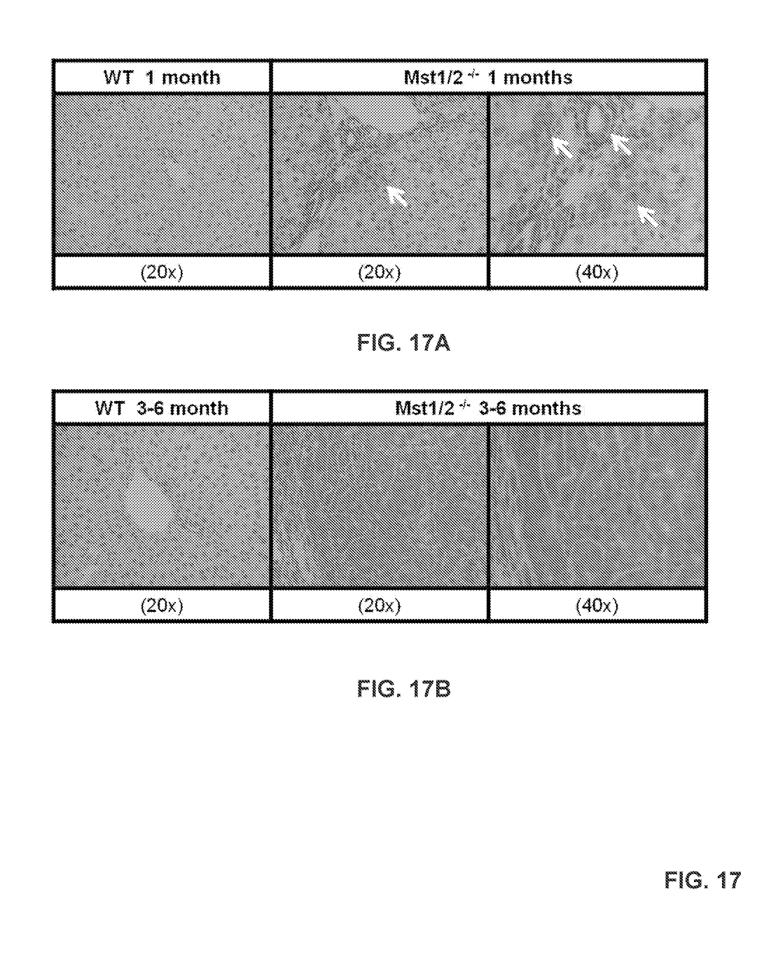

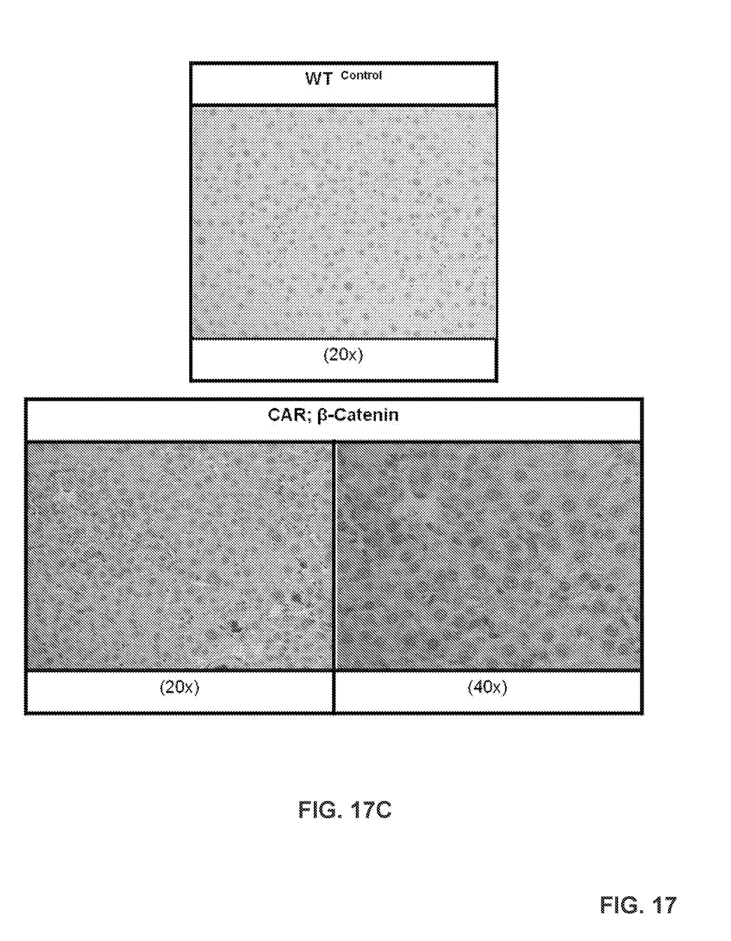

Considering Applicants' previous finding of increased P2X3 expression in patient tumors and HCC cell lines, Applicants analyzed Mst1/2^{-/-} tumor sections using immunohistochemistry (IHC) for P2X3 expression. Applicants found, that in spite of decreased P2X3 mRNA expression, there was increased P2X3 protein expression in Mst1/2^{-/-} tissues compared to age matched WT sections (FIGS. 17A-B). Furthermore, Applicants analyzed P2X3 in another HCC model likely dependent on the hippo pathway. The Constitutive Androstane Receptor, NR1I3 (CAR) activated; β-catenin activated mouse exhibits tumorigenesis of a typical HCC phenotype at 8 months. IHC analysis revealed increased P2X3 expression in CAR/β-catenin mouse sections compared to age-matched WT control mice (FIG. 17C).

### Example 2.9. P2X5 Purinergic Receptor Overexpression Correlates with Lower Overall Survival and Recurrence Free Survival in HCC Patients

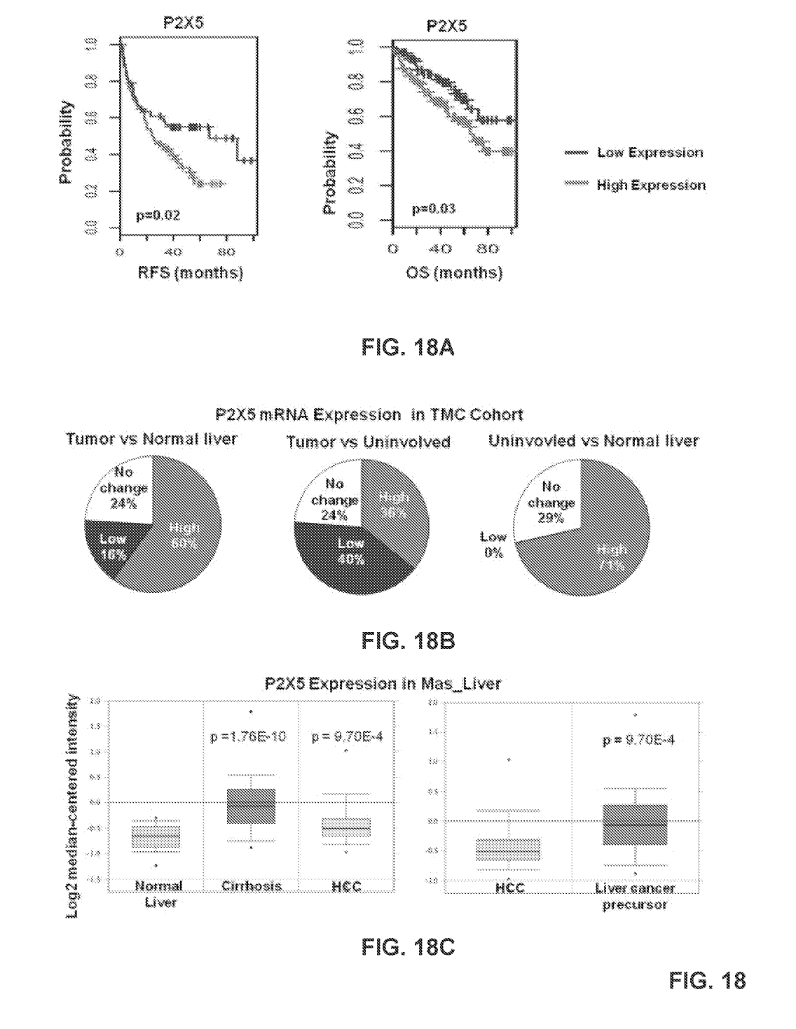

Gene expression profiling was done on Applicants' Korean Cohort tumors (n=188) described recently (Oncotarget 2015; 6:41162-41179). Kaplan-Meier analysis of P2X5 expression determined that P2X5 overexpression is strongly correlated with reduced overall survival (OS) and recurrence free survival (RFS) (FIG. 18A). Applicants have recently reported that 60% of HCC patients had 2-fold or greater P2X5 mRNA expression compared to normal liver tissue (Oncotarget 2015; 6:41162-41179). Only 36% of patients had high P2X5 expression compared to their uninvolved areas, suggesting upregulation in uninvolved areas which are likely inflamed and diseased (Oncotarget 2015; 6:41162-41179). In fact, 71% of HCC patients exhibited high P2X5 expression in their uninvolved areas compared to normal liver tissue (FIG. 18B). Oncomine analysis of the Mas Liver dataset revealed P2X5 overexpression in cirrhotic tissue compared to normal liver (1.5 fold) and P2X5 overexpression in HCC tumor tissue compared to normal liver (1.2 fold) (FIG. 18C). In addition, P2X5 was overexpressed in liver cancer precursor tissue compared to HCC tumor tissue (1.3 fold) (FIG. 18C).

Similarly, 40% of Applicants' TMC cohort HCC patients had P2X5 low expression in tumor compared to uninvolved areas, suggesting higher expression in the surrounding, likely cancer precursor tissue compared to the actual tumor tissue (FIG. 18B). Oncomine analysis of the Wurmbach Liver dataset exhibited similar patterns as seen in the Mas dataset.

### Example 2.10. P2Y2 Purinergic Receptor Underexpression Correlates with Lower Overall Survival and Recurrence Free Survival in HCC Patients

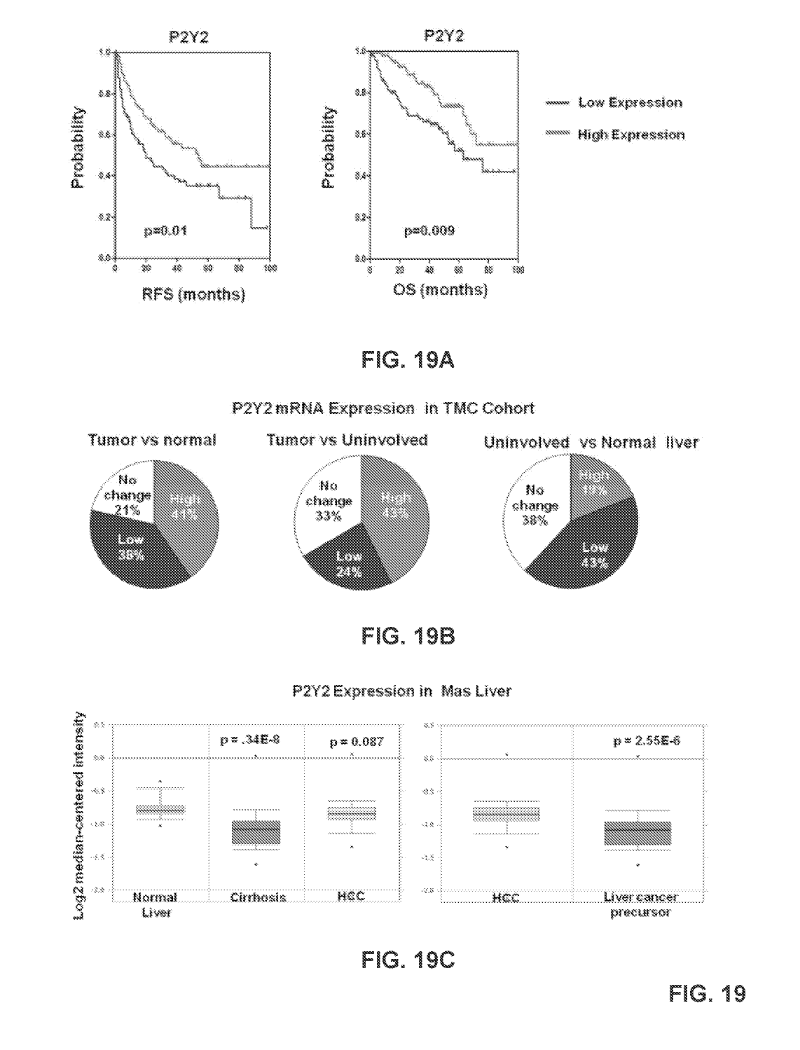

Analysis of Applicants' Korean patient cohort revealed that patients with low P2Y2 expression had a significantly lower overall survival (p=0.009) and recurrence free survival (p=0.01) rate than those patients with high P2Y2 expression (FIG. 19A). Applicants' TMC cohort analysis revealed that P2Y2 was the receptor with the largest number of patients (38%) exhibiting low expression when compared to normal livers. Even more patients (43%) had low expression in their uninvolved tissue compared to normal livers (FIG. 19B). Oncomine analysis showed that P2Y2 is underexpressed in cirrhosis and in HCC compared to normal livers (FIG. 19C). Liver cancer precursor tissues exhibited statistically significant P2Y2 underexpression compared to HCC tumors (FIG. 19C). P2Y2 was underexpressed when compared to 8 other cancer types in the Su multi-cancer data set (p=0.01).

### Example 2.11. Extracellular Nucleotides Activate Hippo Pathway Upstream Kinases

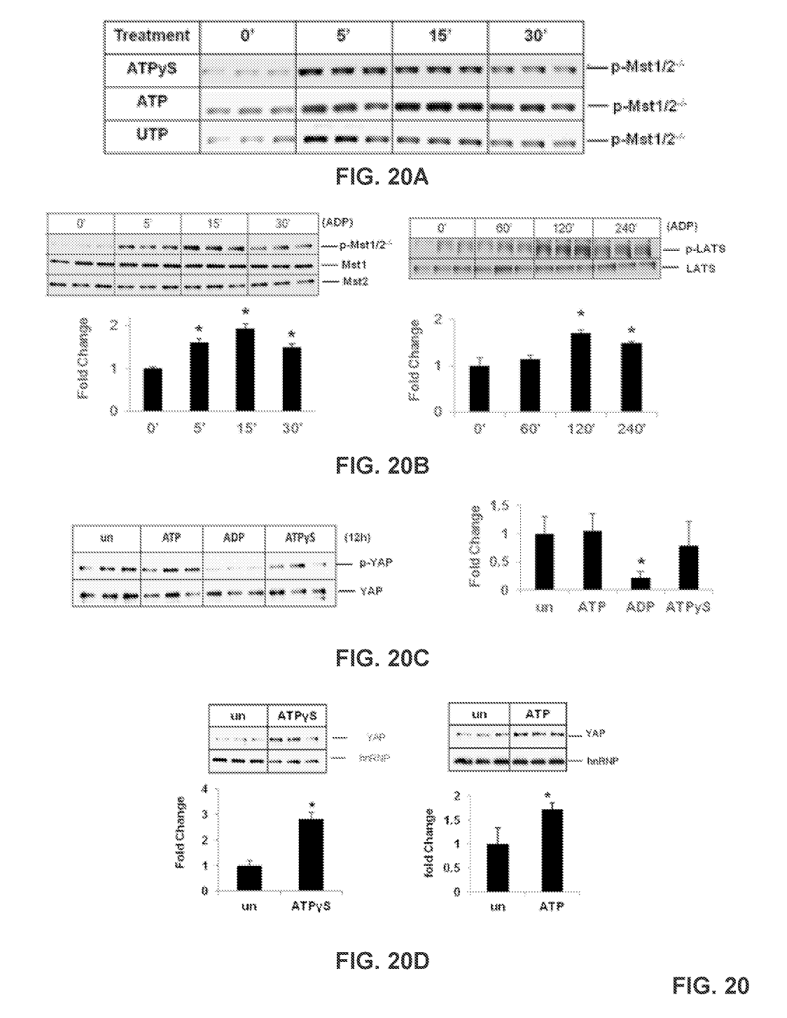

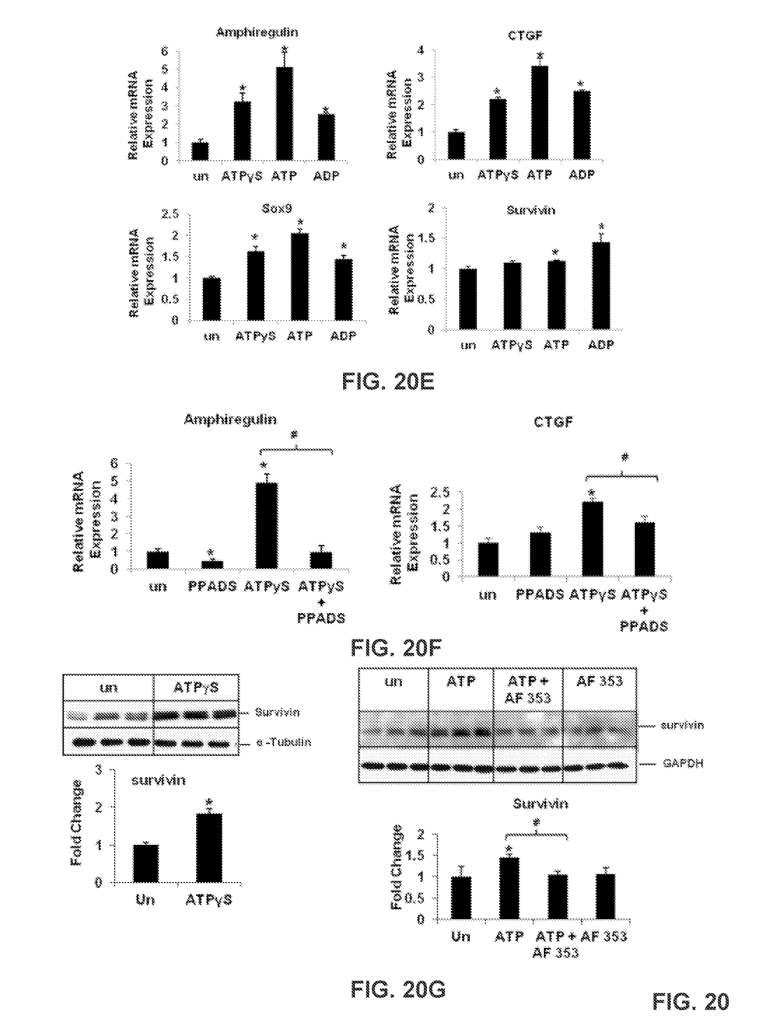

Dysregulation of P2 purinergic receptor mRNA expression was observed in Mst1/2^{-/-} mice as young as 1 mo (P2X1, P2X3, P2X5, P2X6, P2Y1, P2Y4, P2Y6) prior to tumor formation (FIGS. 16A-B). This suggests that manipulation of hippo genes may have a direct effect on P2 purinergic receptors, implicating an interaction between the two pathways. Therefore, Applicants performed in vitro studies to test whether activation of P2 purinergic receptors may affect the hippo pathway and its downstream effectors. Huh7 cells—human derived hepatocellular carcinoma cells—maintained in serum free media for 24 hours—were treated with P2 purinergic receptor agonists. As early as 5 minutes and sustained at 4 hours (data not shown) after ATPγS, ATP or UTP treatment, Mst1/2 phosphorylation was significantly induced (FIG. 20A). ADP treatment also significantly induced Mst1/2 phosphorylation (5 min-1.6 fold, 15 min-1.9 fold, 30 min-1.5 fold). ADP treatment alone was also sufficient to induce LATs phosphorylation (2 h-1.7 fold, 4 h-1.5 fold) (FIG. 20B). These data suggest that extracellular nucleotide treatment alone is sufficient to activate hippo pathway upstream kinases.

### Example 2.12. Extracellular Nucleotides Induce Nuclear Yap Expression and Yap Target Genes

Hippo kinases Mst1/2 and Lats1/2 work through a phosphorylation cascade to phosphorylate Yap, thereby retaining Yap in the cytoplasm. In spite of nucleotide-mediated Mst1/2 and Lats phosphorylation, ATP, ADP and ATPγS treatment was individually insufficient to induce Yap phosphorylation. In fact, 12 h ADP treatment resulted in a significant reduction in Yap phosphorylation (0.2 fold) (FIG. 20C). Furthermore, western blotting of Huh7 nuclear fractions revealed that ATPγS treatment alone was sufficient to increase nuclear YAP protein expression (2.8 fold) compared to untreated cells (FIG. 20D). Similarly, ATP treatment alone increased nuclear YAP protein expression (1.7 fold) (FIG. 20D).

Next, Applicants tested whether extracellular nucleotide treatment may have an effect on YAP target genes important in hepatocellular carcinoma. ATPγS, ATP and ADP individually induced cyclin E (2.0, 2.1, 2.4 fold), AREG (3.3, 5.1, 2.6 fold), CTGF (2.2, 3.4, 2.5 fold), Sox9 (1.6, 2.0, 1.4 fold) and survivin (1.1, 1.1, 1.4 fold) gene expression (FIG. 20E). Broad spectrum P2 purinergic receptor antagonist, PPADS, completely attenuated ATPγS-induced AREG and CTGF expression, confirming that nucleotides induce Yap target genes via the activation of P2 purinergic receptors (FIG. 20F). Western blotting reveals that, in spite of modest nucleotide-mediated mRNA induction, ATPγS treatment alone was sufficient to induce survivin protein expression (1.8 fold). Moreover, P2X3 antagonist (AF-353) pretreatment attenuated ATP mediated induction of survivin (FIG. 20G).

### Example 2.13. High Expression of AREG, CTGF, SOX9 and Survivin Overlaps with High P2 Purinergic Receptor Expression in HCC Tumors

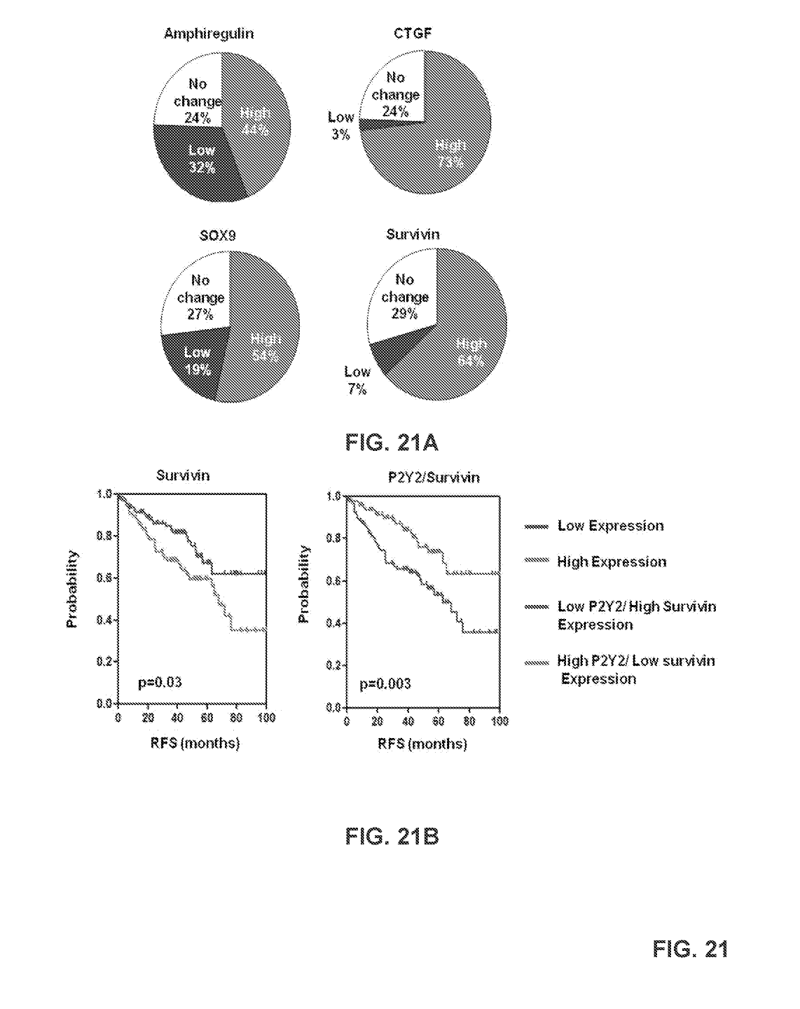

Applicants used qRT-PCR to examine the mRNA expression of YAP target genes in a TMC cohort. Similar to previous reports, Applicants found that the majority of patients had high expression (≥2-fold) of AREG (44%), CTGF (73%), Sox9 (54%) and survivin (64%) in tumors compared to uninvolved livers (FIG. 21). A large frequency of patients with high expression in each of these genes also exhibited high P2 purinergic receptor expression. In particular, high P2X2 receptor expression was observed in 88% of patients with ‘high’ AREG, 77% with ‘high’ CTGF, 83% with ‘high’ Sox9 and 81% with ‘high’ survivin expression. By contrast, lower frequencies of P2Y2 ‘high’ expression were observed among the high expressing YAP target genes. Only 33% of patients with ‘high’ CTGF expression also exhibited high P2Y2 receptor expression (Table 5).

TABLE 5
Frequency of P2 purinergic receptor high expression when YAP
target genes are overexpressed.

| | ↑ AREG | ↑ CTGF | ↑SOX9 | ↑Survivin |
|---|---|---|---|---|
| ↑ P2X2 (%) | 88 | 77 | 83 | 81 |
| ↑ P2X3 (%) | 76 | 80 | 70 | 73 |
| ↑ P2X7 (%) | 71 | 63 | 61 | 62 |
| ↑ P2Y6 (%) | 71 | 80 | 70 | 77 |
| ↑ P2Y14 (%) | 71 | 63 | 57 | 58 |
| ↑ P2X5 (%) | 65 | 60 | 57 | 54 |
| ↑ P2Y4 (%) | 65 | 63 | 65 | 65 |
| ↑ P2X1 (%) | 59 | 50 | 48 | 46 |
| ↑ P2X4 (%) | 59 | 37 | 39 | 38 |
| ↑ P2X6 (%) | 59 | 50 | 52 | 46 |
| ↑ P2Y11 (%) | 53 | 47 | 48 | 46 |
| ↑ P2Y12 (%) | 53 | 43 | 48 | 38 |
| ↑ P2Y13 (%) | 53 | 43 | 48 | 35 |
| ↑ P2Y1 (%) | 47 | 43 | 39 | 38 |
| ↑ P2Y2 (%) | 47 | 33 | 35 | 38 |

### Example 2.14. “High” Survivin with “Low” P2Y2 Correlates with Lower Overall Survival in HCC Patients

Applicants assessed survival in the Korean cohort based on gene expression. Similar to the literature, patients with higher survivin expression had lower survival than those with lower survivin expression (FIG. 21B). None of the other YAP target genes had significant correlations with survival. Moreover, those patients with a high survivin to low P2Y2 expression ratio had significantly lower survival than those with low survivin to high P2Y2 ratio (FIG. 21B).

### Example 2.15. Discussion

Applicants have shown dysregulation of P2 purinergic receptors in two mouse models of HCC. The altered P2 purinergic receptor expression observed in Mst1/2^{-/-} livers compared to WT suggests a role for purinergic signaling in the pathogenesis of HCC. P2X5 mRNA upregulation was most dramatic among the 15 receptor subtypes. Applicants also showed dramatic upregulation of P2X5 in TMC tumors compared to normal livers and confirmed observations with Oncomine analysis of publicly available Mas, Wurmbach and Su Multi-cancer datasets. Furthermore, Applicants show for the first time that high P2X5 expression correlates with significantly lower patient survival.

P2X5 expression is reported in the male reproductive organs with a likely function in epithelial cell differentiation. It is also expressed in a broad range of lymphoid malignancies but its function has not been defined.

P2Y2 has been well studied in various contexts, including cancer biology. P2Y2 receptor is significantly expressed in prostate cancer, breast cancer and HCC cells and implicated in cell proliferation, invasion and metastasis. P2Y2 activation is reported to be apoptotic in nasopharyngeal carcinoma cells, anti-proliferative in metastatic breast cancer cells and reported to promote apoptosis and inhibit growth in colorectal carcinoma cells. Applicants show that P2Y2 receptor is downregulated at the protein level in the Mst1/2^{-/-} HCC mouse model.

Oncomine analysis of the Mas dataset also shows that cirrhotic and liver cancer precursor tissues underexpress P2Y2. Furthermore, Applicants showed that low expression of P2Y2 significantly correlates with lower patient survival. Considering that some 43% of Applicants' TMC cohort tumors had high expression compared to uninvolved areas, it is likely that there are subsets of patients with differential P2Y2 expression.

Applicants have shown for the first time that extracellular nucleotides alone may induce a number of Yap target genes, all implicated in HCC. Applicants also outlined the effect of nucleotides on cell cycle progression in a recent report, highlighting a role for cyclin E in HCC cell proliferation. In this Example, Applicants now show that extracellular nucleotides induce AREG, CTGF, Sox9 and survivin mRNA expression and survivin protein expression. Furthermore, Applicants identified P2X3 as a likely receptor involved in ATP-mediated survivin induction.

Without further elaboration, it is believed that one skilled in the art can, using the description herein, utilize the present disclosure to its fullest extent. The embodiments described herein are to be construed as illustrative and not as constraining the remainder of the disclosure in any way whatsoever. While the embodiments have been shown and described, many variations and modifications thereof can be made by one skilled in the art without departing from the spirit and teachings of the invention. Accordingly, the scope of protection is not limited by the description set out above, but is only limited by the claims, including all equivalents of the subject matter of the claims. The disclosures of all patents, patent applications and publications cited herein are hereby incorporated herein by reference, to the extent that they provide procedural or other details consistent with and supplementary to those set forth herein.

What is claimed is:

1. A method of inhibiting cancer cells, said method comprising:
exposing the cancer cells to a purinergic receptor antagonist,
wherein the purinergic receptor antagonist targets one or more purinergic receptors of the cancer cells.

2. The method of claim 1, wherein the purinergic receptor antagonist is selected from the group consisting of AF-353, A317491, AF-219, Suramin, PPADS, MRS2159, NF449, PSB-1011, NF770, A740003, RB2, MRS2179, MRS2279, MRS2500, MRS2578, NF340, PSB0739, PPTN, and combinations thereof.

3. The method of claim 1, wherein the purinergic receptor antagonist is A317491.

4. The method of claim 1, wherein the purinergic receptor antagonist is AF-353.

5. The method of claim 1, wherein the purinergic receptor antagonist is PPADS

6. The method of claim 1, wherein the one or more purinergic receptors comprise P2 purinergic receptors.

7. The method of claim 1, wherein the one or more purinergic receptors comprise P2X purinergic receptor subtypes.

8. The method of claim 7, wherein the P2X purinergic receptor subtypes are selected from the group consisting of P2X1, P2X2, P2X3, P2X4, P2X5, P2X6, P2X7, and combinations thereof.

9. The method of claim 1, wherein the one or more purinergic receptors comprise P2Y purinergic receptor subtypes.

10. The method of claim 9, wherein the one or more P2Y purinergic receptor subtypes are selected from the group consisting of P2Y1, P2Y2, P2Y4, P2Y6, P2Y11, P2Y12, P2Y13, P2Y14, and combinations thereof.

11. The method of claim 1, wherein the one or more purinergic receptors comprise P2X5.

12. The method of claim 1, wherein the one or more purinergic receptors comprise P2X3.

13. The method of claim 1, wherein the one or more purinergic receptors comprise P2Y2.

14. The method of claim 1, wherein the cancer cells are associated with liver tumors.

15. The method of claim 1, wherein the cancer cells are associated with hepatocellular carcinoma.

16. The method of claim 1, wherein the exposing occurs by incubating the cancer cells with the purinergic receptor antagonist.

17. The method of claim 1, wherein the exposing occurs in vitro.

18. The method of claim 1, wherein the exposing occurs in vivo in a subject.

19. The method of claim 18, wherein the exposing occurs by administering the purinergic receptor antagonist to the subject.

20. The method of claim 19, wherein the administering occurs by a method selected from the group consisting of oral administration, inhalation, subcutaneous administration, intravenous administration, intraperitoneal administration, intramuscular administration, intrathecal injection, topical administration, central administration, peripheral administration, and combinations thereof.

21. The method of claim 18, wherein the subject is a human being.

22. The method of claim 21, wherein the subject is suffering from hepatocellular carcinoma.

23. The method of claim 22, wherein the method is utilized to treat the hepatocellular carcinoma in the subject.

24. The method of claim 1, wherein the exposing kills the cancer cells.

25. The method of claim 1, wherein the exposing reduces or inhibits the proliferation of the cancer cells.

26. The method of claim 1, wherein the exposing attenuates ATP-mediated protein expression in the cancer cells.

27. The method of claim 26, wherein the attenuated proteins are selected from the group consisting of cyclin D3, cyclin E, cyclin A, Amphiregulin, CTGF, Sox 9, Survivin, and combinations thereof.

28. A method of treating hepatocellular carcinoma in a subject, said method comprising:
administering a purinergic receptor antagonist to the subject,
wherein the purinergic receptor antagonist targets one or more purinergic receptors of hepatocellular carcinoma cells in the subject.

29. The method of claim 28, wherein the purinergic receptor antagonist is selected from the group consisting of AF-353, A317491, AF-219, Suramin, PPADS, MRS2159, NF449, PSB-1011, NF770, A740003, RB2, MRS2179, MRS2279, MRS2500, MRS2578, NF340, PSB0739, PPTN, and combinations thereof.

30. The method of claim 28, wherein the purinergic receptor antagonist is A317491.

31. The method of claim 28, wherein the purinergic receptor antagonist is AF-353.

32. The method of claim 28, wherein the purinergic receptor antagonist is PPADS

33. The method of claim 28, wherein the one or more purinergic receptors comprise P2 purinergic receptors.

34. The method of claim 28, wherein the one or more purinergic receptors comprise P2X purinergic receptor subtypes.

35. The method of claim 34, wherein the P2X purinergic receptor subtypes are selected from the group consisting of P2X1, P2X2, P2X3, P2X4, P2X5, P2X6, P2X7, and combinations thereof.

36. The method of claim 28, wherein the one or more purinergic receptors comprise P2Y purinergic receptor subtypes.

37. The method of claim 36, wherein the one or more P2Y purinergic receptor subtypes are selected from the group consisting of P2Y1, P2Y2, P2Y4, P2Y6, P2Y11, P2Y12, P2Y13, P2Y14, and combinations thereof.

38. The method of claim 28, wherein the one or more purinergic receptors comprise P2X5.

39. The method of claim 28, wherein the one or more purinergic receptors comprise P2X3.

40. The method of claim 28, wherein the one or more purinergic receptors comprise P2Y2.

41. The method of claim 28, wherein the hepatocellular carcinoma cells are associated with liver tumors.

42. The method of claim 28, wherein the administering occurs by a method selected from the group consisting of oral administration, inhalation, subcutaneous administration, intravenous administration, intraperitoneal administration, intramuscular administration, intrathecal injection, topical administration, central administration, peripheral administration, and combinations thereof.

43. The method of claim 28, wherein the subject is a human being.

44. The method of claim 28, wherein the administering kills the hepatocellular carcinoma cells.

45. The method of claim 28, wherein the administering reduces or inhibits the proliferation of the hepatocellular carcinoma cells.

46. The method of claim 28, wherein the administering attenuates ATP-mediated protein expression in the hepatocellular carcinoma cells.

47. The method of claim 46, wherein the attenuated proteins are selected from the group consisting of cyclin D3, cyclin E, cyclin A, Amphiregulin, CTGF, Sox 9, Survivin, and combinations thereof.
